(19) United States
(12) Patent Application Publication
(10) Pub. No.: US 2025/0316388 A1
(43) Pub. Date: Oct. 9, 2025

(54) METHOD AND APPARATUS FOR PREDICTING DEGENERATIVE BRAIN FUNCTION DECLINE AND COGNITIVE IMPAIRMENT USING VISION LANGUAGE MODEL AND GRAPH NEURAL NETWORK

(71) Applicant: Electronics and Telecommunications Research Institute, Daejeon (KR)

(72) Inventors: Byounghwa LEE, Daejeon (KR); Byung Ok KANG, Daejeon (KR); Jeonguk BANG, Daejeon (KR)

(73) Assignee: Electronics and Telecommunications Research Institute, Daejeon (KR)

(21) Appl. No.: 19/242,069

(22) Filed: Jun. 18, 2025

(30) Foreign Application Priority Data
Jun. 19, 2024 (KR) 10-2024-0079454
Jun. 16, 2025 (KR) 10-2025-0078633

(51) Int. Cl. G16H 50/20; G06V 10/74; G06V 10/764; G06V 10/82; G10L 15/04; G10L 15/183; G10L 25/30; G10L 25/66

(52) U.S. Cl. CPC G16H 50/20; G06V 10/761; G06V 10/764; G06V 10/82; G10L 15/04; G10L 15/183; G10L 25/30; G10L 25/66

(57) ABSTRACT

Disclosed are a method and apparatus for predicting a degenerative brain function decline and a cognitive impairment using a vision language model and a graph neural network. In order to diagnose a degenerative brain function decline or a cognitive impairment, a subject is required to describe a situation that appears in a given image by speech. An embodiment of the present disclosure proposes a method and apparatus for generating a graph indicative of a relation between each part of the image and contents of the part, which are described by a subject, by using a vision language model and determining or predicting whether the subject has a degenerative brain function decline or a cognitive impairment by using a graph neural network.

### CROSS-REFERENCE TO RELATED APPLICATION

This application claims priority under 35 U.S.C. § 119 to Korean Patent Application Nos. 10-2024-0079454, filed on Jun. 19, 2024, and 10-2025-0078633, Jun. 16, 2025, the disclosure of which is incorporated herein by reference in its entirety.

### BACKGROUND

### 1. Technical Field

The present disclosure corresponds to a technical field in which a disease is diagnosed or predicted by using an artificial intelligence (AI) model based on an image and speech data.

### 2. Related Art

As the elderly population is globally increased, degenerative brain function decline patients and cognitive impairment patients are greatly increased. In particular, it is very important to early detect dementia, such as Alzheimer's disease. The reason for this is that Alzheimer's disease starts from a mild cognitive impairment (MCI) at an early stage, its state gradually become degenerated, and develops into dementia. Accordingly, it is necessary to check a longitudinal trend through periodic tests. To this end, a cognitive function is evaluated through a picture description task.

The picture description task is a task in which a subject describes the situation of a given picture through an utterance while watching the given picture. A degenerative brain function decline patient or a cognitive impairment patient does not properly cognize or does not utter each situation of a given picture or does not describe each situation of a given picture through a language having a meaning. That is, the brain function and cognitive function of the subject may be evaluated based on the description ability of the subject for a situation shown in the picture.

The existing degenerative brain function decline and cognitive impairment prediction model through a picture description task includes an aspect in which only a speech utterance of a subject and text transcribed from the speech of the subject are considered. That is, the existing model has a problem in that an error occurs in determining that there is no problem with the brain function and cognitive function of a subject when the subject describes a wrong situation in a logical language and a fluent utterance even in the case of the wrong situation that is not present in a given picture.

Furthermore, a subject who has a normal cognitive function may not describe a situation which may be sufficiently described by not cognizing the situation. The existing method using only speech and text has a problem in that such a case (i.e., a case in which a subject does not cognize a situation) is not properly detected.

Furthermore, most of the existing prediction models are black box models, and do not overcome the limit of deep learning in which grounds for prediction are not properly described.

### SUMMARY

Various embodiments are directed to providing a method and apparatus for predicting a degenerative brain function decline and a cognitive impairment, which transcribe speech uttered by a subject in order to describe a given image into text, generate a graph indicative of relation between the image and the text by using a vision language model (VLM), and determine or predict whether the subject has a mild cognitive impairment or dementia by using a pre-trained graph neural network (GNN).

An object of the present disclosure is not limited to the aforementioned object, and other objects not described above may be evidently understood by those skilled in the art from the following description.

According to an embodiment of the present disclosure, a method performed by an apparatus for predicting a degenerative brain function decline and a cognitive impairment includes receiving a target image of a picture description task and utterance speech data of a subject for the target image, generating a sub-image embedding vector that is an embedding vector of a sub-image of the target image, extracting one or more sentences by segmenting utterance text that is generated by transcribing the utterance speech data in a sentence unit and generating a sentence embedding vector that is an embedding vector of the sentence, and calculating similarity between the sub-image and the sentence by using a vision language model and determining that the subject corresponds to any one of a degenerative brain function decline and cognitive impairment group and a normal group based on the sub-image embedding vector, the sentence embedding vector, and the similarity.

The generating of the sub-image embedding vector may include generating the sub-image embedding vector by using the vision language model.

The generating of the sentence embedding vector may include generating the sentence embedding vector by using the vision language model.

The determining of that the subject corresponds to any one of the degenerative brain function decline and cognitive impairment group and the normal group may include calculating the similarity between the sub-image and the sentence by using the vision language model, generating a bipartite graph including a sub-image node corresponding to the sub-image embedding vector and a sentence node corresponding to the sentence embedding vector, wherein a weight of an edge that connects the sub-image node and the sentence node is set in the bipartite graph based on the similarity, inputting the bipartite graph to a graph neural network and generating a graph-level embedding vector through information propagation, and calculating a probability that the subject is to belong to the normal group by inputting the graph-level embedding vector to a classifier based on a pre-trained artificial neural network and classifying the subject as any one group of the degenerative brain function decline and cognitive impairment group and the normal group based on the probability.

The graph neural network may be a graph convolution neural network.

The method may further include extracting a speech feature of the subject from the utterance speech data and generating a speech embedding vector based on the speech feature.

The classifying of the subject may include calculating the probability by inputting the graph-level embedding vector and the speech embedding vector to the classifier.

The method may further include extracting a text feature by inputting the utterance text to a language model and generating the text embedding vector based on the text feature.

The classifying of the subject may include calculating the probability by inputting the graph-level embedding vector and the text embedding vector to the classifier.

The method may further include generating a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed, calculating first similarity between the first representative embedding vector and the sentence embedding vector of the degenerative brain function decline and cognitive impairment group and generating a similarity high-rank sentence group by grouping sentence embedding vectors corresponding to a predetermined high-rank percentage, among all of the sentence embedding vectors of the degenerative brain function decline and cognitive impairment group, based on the first similarity, calculating second similarity between the second representative embedding vector and the sentence embedding vector of the degenerative brain function decline and cognitive impairment group and generating a similarity low-rank sentence group by grouping sentence embedding vectors corresponding to a predetermined low-rank percentage, among all of the sentence embedding vectors of the degenerative brain function decline and cognitive impairment group, based on the second similarity, and selecting representative sentences corresponding to the similarity high-rank sentence group and the similarity low-rank sentence group, respectively, based on a predetermined reference and displaying the representative sentences through an output interface device.

The method may further include generating a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed, calculating first similarity between the first representative embedding vector and the sentence embedding vector of the degenerative brain function decline and cognitive impairment group, generating a first relevant sentence set by grouping sentence embedding vectors corresponding to a predetermined high-rank percentage, among all of the sentence embedding vectors of the degenerative brain function decline and cognitive impairment group, based on the first similarity, and generating a first irrelevant sentence set by grouping sentence embedding vectors corresponding to a predetermined low-rank percentage, among all of the sentence embedding vectors of the degenerative brain function decline and cognitive impairment group, based on the first similarity, calculating second similarity between the second representative embedding vector and the sentence embedding vector of the degenerative brain function decline and cognitive impairment group, generating a second relevant sentence set by grouping sentence embedding vectors corresponding to a predetermined low-rank percentage, among all of the sentence embedding vectors of the degenerative brain function decline and cognitive impairment group, based on the second similarity, and generating a second irrelevant sentence set by grouping sentence embedding vectors corresponding to a predetermined high-rank percentage, among all of the sentence embedding vectors of the degenerative brain function decline and cognitive impairment group, based on the second similarity, calculating third similarity between the second representative embedding vector and the sentence embedding vector of the normal group, generating a third relevant sentence set by grouping sentence embedding vectors corresponding to a predetermined high-rank percentage, among all of the sentence embedding vectors of the normal group, based on the third similarity, and generating a third irrelevant sentence set by grouping sentence embedding vectors corresponding to a predetermined low-rank percentage, among all of the sentence embedding vectors of the normal group, based on the third similarity, calculating fourth similarity between the first representative embedding vector and the sentence embedding vector of the normal group, generating a fourth relevant sentence set by grouping sentence embedding vectors corresponding to a predetermined low-rank percentage, among all of the sentence embedding vectors of the normal group, based on the fourth similarity, and generating a fourth irrelevant sentence set by grouping sentence embedding vectors corresponding to the predetermined high-rank percentage, among all of the sentence embedding vectors of the normal group, based on the fourth similarity, selecting a word not included in the third relevant sentence set, among words included in the first relevant sentence set, and setting the selected word as a first keyword, selecting a word not included in the fourth relevant sentence set, among words included in the second relevant sentence set, and setting the selected word as a second keyword, selecting a word not included in the third irrelevant sentence set, among words included in the first irrelevant sentence set, and setting the selected word as a third keyword, selecting a word not included in the fourth irrelevant sentence set, among words included in the second irrelevant sentence set, and setting the selected word as a fourth keyword, and outputting the first keyword and the second keyword as keyword that help in determining the degenerative brain function decline and cognitive impairment group and outputting the third keyword and the fourth keyword as keywords that do not help in determining the degenerative brain function decline and cognitive impairment group.

The method may further include calculating the probability for an identical subject with respect to different target images at regular time intervals during a predetermined period by a predetermined number of times and inputting the probability calculated during the predetermined period to a pre-trained longitudinal analysis model and determining that the subject is to belong to the degenerative brain function decline and cognitive impairment group after a predetermined period based on an output of the longitudinal analysis model.

An apparatus for predicting a degenerative brain function decline and a cognitive impairment includes a processor and memory in which one or more instructions executed by the processor are stored.

The one or more instructions may include an instruction to receive a target image of a picture description task and utterance speech data of a subject for the target image, an instruction to generate a sub-image embedding vector that is an embedding vector of a sub-image of the target image, an instruction to extract one or more sentences by segmenting utterance text that is generated by transcribing the utterance speech data in a sentence unit and to generate a sentence embedding vector that is an embedding vector of the sentence, and an instruction to calculate similarity between the sub-image and the sentence by using a vision language model and to determine that the subject corresponds to any one of a degenerative brain function decline and cognitive impairment group and a normal group based on the sub-image embedding vector, the sentence embedding vector, and the similarity.

The instruction to generate the sub-image embedding vector may include an instruction to generate the sub-image embedding vector by using the vision language model.

The instruction to generate the sentence embedding vector may include an instruction to generate the sentence embedding vector by using the vision language model.

The instruction to determine that the subject corresponds to any one of the degenerative brain function decline and cognitive impairment group and the normal group may include an instruction to calculate the similarity between the sub-image and the sentence by using the vision language model, an instruction to generate a bipartite graph including a sub-image node corresponding to the sub-image embedding vector and a sentence node corresponding to the sentence embedding vector, wherein a weight of an edge that connects the sub-image node and the sentence node is set in the bipartite graph based on the similarity, an instruction to input the bipartite graph to a graph neural network and to generate a graph-level embedding vector through information propagation, and an instruction to calculate a probability that the subject is to belong to the normal group by inputting the graph-level embedding vector to a classifier based on a pre-trained artificial neural network and to classify the subject as any one group of the degenerative brain function decline and cognitive impairment group and the normal group based on the probability.

The graph neural network may be a graph convolution neural network.

The one or more instructions may further include an instruction to extract a speech feature of the subject from the utterance speech data and an instruction to generate a speech embedding vector based on the speech feature.

The instruction to classify the subject may include an instruction to calculate the probability by inputting the graph-level embedding vector and the speech embedding vector to the classifier.

The one or more instructions may further include an instruction to extract a text feature by inputting the utterance text to a language model and an instruction to generate the text embedding vector based on the text feature.

The instruction to classify the subject may include an instruction to calculate the probability by inputting the graph-level embedding vector and the text embedding vector to the classifier.

The one or more instructions may further include an instruction to generate a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed, an instruction to calculate first similarity between the first representative embedding vector and the sentence embedding vector of the degenerative brain function decline and cognitive impairment group and to generate a similarity high-rank sentence group by grouping sentence embedding vectors corresponding to a predetermined high-rank percentage, among all of the sentence embedding vectors of the degenerative brain function decline and cognitive impairment group, based on the first similarity, an instruction to calculate second similarity between the second representative embedding vector and the sentence embedding vector of the degenerative brain function decline and cognitive impairment group and generating a similarity low-rank sentence group by grouping sentence embedding vectors corresponding to a predetermined low-rank percentage, among all of the sentence embedding vectors of the degenerative brain function decline and cognitive impairment group, based on the second similarity, and an instruction to select representative sentences corresponding to the similarity high-rank sentence group and the similarity low-rank sentence group, respectively, based on a predetermined reference and displaying the representative sentences through an output interface device.

The one or more instructions may further include an instruction to generate a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed, an instruction to calculate first similarity between the first representative embedding vector and the sentence embedding vector of the degenerative brain function decline and cognitive impairment group, to generate a first relevant sentence set by grouping sentence embedding vectors corresponding to a predetermined high-rank percentage, among all of the sentence embedding vectors of the degenerative brain function decline and cognitive impairment group, based on the first similarity, and to generate a first irrelevant sentence set by grouping sentence embedding vectors corresponding to a predetermined low-rank percentage, among all of the sentence embedding vectors of the degenerative brain function decline and cognitive impairment group, based on the first similarity, an instruction to calculate second similarity between the second representative embedding vector and the sentence embedding vector of the degenerative brain function decline and cognitive impairment group, to generate a second relevant sentence set by grouping sentence embedding vectors corresponding to a predetermined low-rank percentage, among all of the sentence embedding vectors of the degenerative brain function decline and cognitive impairment group, based on the second similarity, and to generate a second irrelevant sentence set by grouping sentence embedding vectors corresponding to a predetermined high-rank percentage, among all of the sentence embedding vectors of the degenerative brain function decline and cognitive impairment group, based on the second similarity, an instruction to calculate third similarity between the second representative embedding vector and the sentence embedding vector of the normal group, to generate a third relevant sentence set by grouping sentence embedding vectors corresponding to a predetermined high-rank percentage, among all of the sentence embedding vectors of the normal group, based on the third similarity, and to generate a third irrelevant sentence set by grouping sentence embedding vectors corresponding to a predetermined low-rank percentage, among all of the sentence embedding vectors of the normal group, based on the third similarity, an instruction to calculate fourth similarity between the first representative embedding vector and the sentence embedding vector of the normal group, to generate a fourth relevant sentence set by grouping sentence embedding vectors corresponding to a predetermined low-rank percentage, among all of the sentence embedding vectors of the normal group, based on the fourth similarity, and to generate a fourth irrelevant sentence set by grouping sentence embedding vectors corresponding to the predetermined high-rank percentage, among all of the sentence embedding vectors of the normal group, based on the fourth similarity, an instruction to select a word not included in the third relevant sentence set, among words included in the first relevant sentence set, and to set the selected word as a first keyword, to select a word not included in the fourth relevant sentence set, among words included in the second relevant sentence set, and to set the selected word as a second keyword, to select a word not included in the third irrelevant sentence set, among words included in the first irrelevant sentence set, and to set the selected word as a third keyword, to select a word not included in the fourth irrelevant sentence set, among words included in the second irrelevant sentence set, and to set the selected word as a fourth keyword, and an instruction to output the first keyword and the second keyword as keyword that help in determining the degenerative brain function decline and cognitive impairment group and to output the third keyword and the fourth keyword as keywords that do not help in determining the degenerative brain function decline and cognitive impairment group.

The one or more instructions may further include an instruction to calculate the probability for an identical subject with respect to different target images at regular time intervals during a predetermined period by a predetermined number of times and an instruction to input the probability calculated during the predetermined period to a pre-trained longitudinal analysis model and to determine that the subject is to belong to the degenerative brain function decline and cognitive impairment group after a predetermined period based on an output of the longitudinal analysis model.

According to an embodiment of the present disclosure, whether a subject has a degenerative brain function decline or a cognitive impairment can be determined based on an image and utterance speech data of the subject that describes the image.

Furthermore, according to an embodiment of the present disclosure, a sentence or a keyword, that is, an important clue in the determination or prediction of a degenerative brain function decline and a cognitive impairment, can be obtained through a graph neural network.

Furthermore, according to an embodiment of the present disclosure, it is possible to early predict a degenerative brain function decline or cognitive impairment of a subject based on longitudinal analysis of the results of the determination of the degenerative brain function decline and the cognitive impairment.

Effects of the present disclosure which may be obtained in the present disclosure are not limited to the aforementioned effects, and other effects not described above may be evidently understood by a person having ordinary knowledge in the art to which the present disclosure pertains from the following description.

### BRIEF DESCRIPTION OF THE DRAWINGS

FIG. 1 is a block diagram illustrating a construction of an apparatus for predicting a degenerative brain function decline and a cognitive impairment according to an embodiment of the present disclosure.

FIG. 2 is a diagram illustrating an example of input data and output data of the apparatus for predicting a degenerative brain function decline and a cognitive impairment.

FIG. 3 is a flowchart for describing a method of predicting a degenerative brain function decline and a cognitive impairment according to a first embodiment of the present disclosure.

FIGS. 4A to 4C are diagrams for describing a process of generating a bipartite graph indicative of a relation between an image and text.

FIG. 5 is a flowchart for describing a method of predicting a degenerative brain function decline and a cognitive impairment according to a second embodiment of the present disclosure.

FIG. 6 is a flowchart for describing a method of displaying representative sentences of a normal group and a degenerative brain function decline group through an explainable model for the prediction of a degenerative brain function decline and a cognitive impairment according to an embodiment of the present disclosure.

FIGS. 7A to 7D are diagrams illustrating examples of representative sentences of a normal group and a degenerative brain function decline group, which are derived through a similarity-based comparison and a dissimilarity-based comparison.

FIG. 8 is a flowchart for describing a method of displaying keywords of a normal group and a degenerative brain function decline group through the explainable model for the prediction of a degenerative brain function decline and a cognitive impairment according to an embodiment of the present disclosure.

FIGS. 9A and 9B are diagrams illustrating examples of keywords of a normal group and a degenerative brain function decline group, which are derived through a similarity-based comparison and a dissimilarity-based comparison.

FIG. 10 is a diagram for describing a longitudinal analysis method for the early prediction of a degenerative brain function decline and a cognitive impairment.

### DETAILED DESCRIPTION

In order to diagnose a degenerative brain function decline and a cognitive impairment, a method of evaluating a cognitive function through a picture description task is performed.

The existing degenerative brain function decline and cognitive impairment prediction model through a picture description task includes an aspect in which only a speech utterance of a subject and text transcribed from the speech of the subject are considered. That is, the existing model has a problem in that it is determined that there is no problem with the brain function and cognitive function of a subject when the subject describes a wrong situation in a logical language and a fluent utterance even in the case of the wrong situation that is not present in a given picture.

Furthermore, a subject who has a normal cognitive function may not describe a situation which may be sufficiently described by not cognizing the situation. The existing method using only speech and text has a problem in that such a case is not properly detected.

In order to overcome the problems, it is necessary to consider an image that is used in a picture description task as an input to the degenerative brain function decline and cognitive impairment prediction model. Furthermore, the degenerative brain function decline and cognitive impairment prediction model may overcome the problems only when the degenerative brain function decline and cognitive impairment prediction model can determine a relation between an image, a subject speech, and text.

In an embodiment of the present disclosure, a relation between each portion of a picture and a sentence that describes the portion is stored in a bipartite graph form by using a vision language model (VLM). Whether a subject has a degenerative brain function decline and a cognitive impairment is predicted by using a graph neural network (GNN) based on a bipartite graph. A prediction model according to an embodiment of the present disclosure has an edge over the existing technology because the prediction model can secure high accuracy compared to the existing prediction model although the prediction model is trained based on existing benchmark validation data.

Furthermore, it is very important to early predict a degenerative brain function decline and a cognitive function decline. In the state in which the cognitive function has been declined, the cognitive function is rarely recovered although the cognitive function is trained. Accordingly, in order to early predict the degenerative brain function decline and the cognitive function decline, an embodiment of the present disclosure proposes longitudinal analysis. That is, according to an embodiment of the present disclosure, a subject can obtain a cognitive function score over time by allowing the subject to describe various pictures during a sufficient period. Furthermore, there is an advantage in that a cognitive function decline of the subject can be early detected by analyzing time-series data that are obtained as described above.

Furthermore, from the viewpoint of an investigator, it is important to check whether there is a degenerative brain function decline and a problem with a cognitive function when a subject commonly tells a sentence and says a keyword in a picture description task.

The existing prediction model is a black box model, and has the limit of deep learning in which the results of prediction are not properly described. In contrast, an embodiment of the present disclosure proposes a method of capturing the use of characteristic sentences and characteristic keywords of subjects who have a degenerative brain function decline and a cognitive function decline by using an embedding vector trained by the GNN. Such a method may be implemented based on the results of the calculation of cosine similarity between the vectors of several groups.

Advantages and characteristics of the present disclosure and a method for achieving the advantages and characteristics will become apparent from embodiments described in detail later in conjunction with the accompanying drawings. However, the present disclosure is not limited to the disclosed embodiments, but may be implemented in various different forms. The embodiments are merely provided to complete the present disclosure and to fully notify a person having ordinary knowledge in the art to which the present disclosure pertains to the category of the present disclosure. The present disclosure is merely defined by the category of the claims. Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other components, steps, operations and/or components in addition to mentioned components, steps, operations and/or components.

Terms, such as a first and a second, may be used to describe various components, but the components should not be restricted by the terms. The terms may be used to only distinguish one component from the other components. Accordingly, a first component may be named a second component without departing from the scope of a right of the present disclosure. Likewise, a second component may also be named a first component.

When it is described that one component is “connected” or “coupled” to the other component, it should be understood that one component may be directly connected or coupled to the other component, but a third component may exist between the two components. In contrast, when it is described that one component is “directly connected to” or “directly coupled to” the other component, it should be understood that a third component does not exist between the two components. Other expressions for describing relations between components, that is, “between ˜”, “just between ˜”, “adjacent to ˜”, and “neighboring ˜”, should be likewise construed.

The followings are a list of abbreviations that are used in embodiments of the present disclosure.

### Abbreviations

AD: Alzheimer's disease
MCI: mild cognitive impairment
HC: healthy control
VLM: vision language model
GNN: graph neural network
CNN: convolutional neural network
GCN: graph convolutional network

Hereinafter, major techniques that are used in embodiments of the present disclosure are described.

### Vision Language Model (VLM)

The VLM has an object of improving performance of a downstream vision and a language task by pre-learning wide image-text pairs. Contrastive language-image pre-training (CLIP), that is, one of the VLMs, uses contrastive learning in which similarity between embeddings generated by a separate encoder that is applied to text and image inputs is calculated and an item having the highest similarity is selected as a text label. Furthermore, an align image and text representations before fusing (ALBEF) aligns image and text expressions before the image and text expressions are connected to a multi-modal encoder with the help of momentum distillation. In general, VLMs, such as CLIP and ALBEF, pre-learns image-text pairs that are collected in a web. CLIP and ALBEF have a disadvantage in that they are vulnerable to noise because many noises are included in web text.

In contrast, bootstrapping language-image pre-training (BLIP) uses a web dataset more effectively by introducing a captioner+filter (CapFilt). In BLIP, a multi-modal encoder-decoder (MED) and the CapFilt contribute to a model viewpoint and a data viewpoint, respectively. BLIP-2, that is, a subsequent model, proposes a more common and calculation-efficient VLM by bootstrapping an off-the-shelf preliminary learning vision model and a language model.

In an embodiment of the present disclosure, various VLMs may be used. For example, in an embodiment of the present disclosure, cosine similarity between each crop image and each sentence may be extracted by using BLIP that is a representative VLM. If a sentence well describes a crop image, the sentence has high cosine similarity with respect to the crop image. As a result of experiments through actual sample sentences, it was found that BLIP accurately calculates similarity between an image and text by well incorporating relevance between an image and a sentence. The image-text cosine similarity is calculated with respect to all of image-text pairs, which becomes an edge weight of a bipartite graph. That is, a cosine similarity matrix becomes an adjacency matrix from the viewpoint of a graph. A node of the bipartite graph includes an image node and a text node. The image node has the embedding vector of a crop image as its attribute. The text node has the embedding vector of a sentence as its attribute. One subject is formed in the form of one bipartite graph. For reference, a subject corresponds to a subject (e.g., a patient).

### Graph Neural Network (GNN)

The GNN is an artificial neural network for processing graph data. If a convolutional neural network (CNN) and the GNN are made to correspond to each other, a pixel of an image in the CNN corresponds to a node of a graph in the GNN. In the GNN, only connected adjacent nodes are considered as if an operation is performed in only adjacent pixels in the CNN. A core of the GNN is message passing or propagation in which embedding is updated by exchanging information with an adjacent node. A graph convolution network (GCN) is a special case of the GNN, and is a method of applying a convolution operation of the CNN to a graph in which aggregation is performed based on information of an adjacent node. Today, most of pieces of GNN architecture are based on the GCN. In this case, as in GraphSAGE, a model using a method of applying a learnable aggregation function also includes a GNN model not a GCN model. In an embodiment of the present disclosure, the GCN model may be used as a graph neural network. For example, as in Equation 1, a GCN model in which edge weights are considered may be used.

$${x}_{i}^{l} = {W}_{1}^{l} {x}_{i}^{l - 1} + {W}_{2}^{l} {Σ}_{j ∈ 𝒩 ( i )} {e}_{j , i} \cdot {x}_{j}^{l - 1} \qquad ( 1 )$$

In Equation 1, xᵢ^{l} and xᵢ^{l-1} are the embedding vectors of an I-th layer and (1-1)-th layer of a node i. In this case, the “node” may be a crop image node or a sentence node. N(i) is a set of adjacent nodes of the node i. e_{j,i} is the weights of an edge that connects a source node j and a target node i. In an embodiment of the present disclosure, image-text similarity that is obtained in a vision language model (VLM) is used as the weight of an edge. Furthermore, W₁^{l} and W₂^{l} is a matrix of learnable parameters from data. Node embedding into which information on a relation between an image and text has been incorporated may be updated through the GCN. A user may set a maximum number I of layers of the GCN. If common ADReSSo data are applied, in most cases, the best performance is obtained when the maximum number I of layers is set to 3 and training is performed. In this case, the setting of the maximum number I of layers may be different depending on the number of data samples. If the maximum number I of layers is excessively great, training is not properly performed because the values of all of node embeddings become similar due to over-smoothing. In contrast, if the maximum number I of layers is small like 1 or 2, there is a problem in that adjacent information is not sufficiently incorporated upon training.

In describing the present disclosure, a detailed description of a related known technology will be omitted if it is deemed to make the subject matter of the present disclosure unnecessarily vague.

Hereinafter, embodiments of the present disclosure are described in detail with reference to the accompanying drawings. In describing the present disclosure, in order to facilitate general understanding of the present disclosure, the same reference numeral is used for the same mean regardless of the reference numeral.

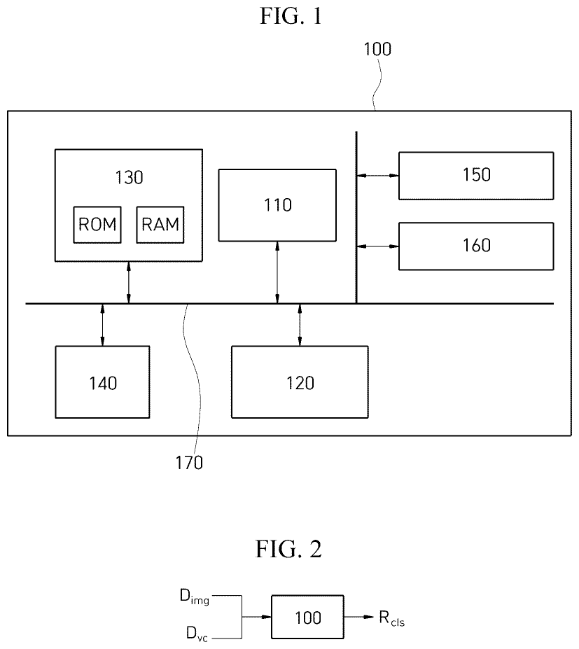

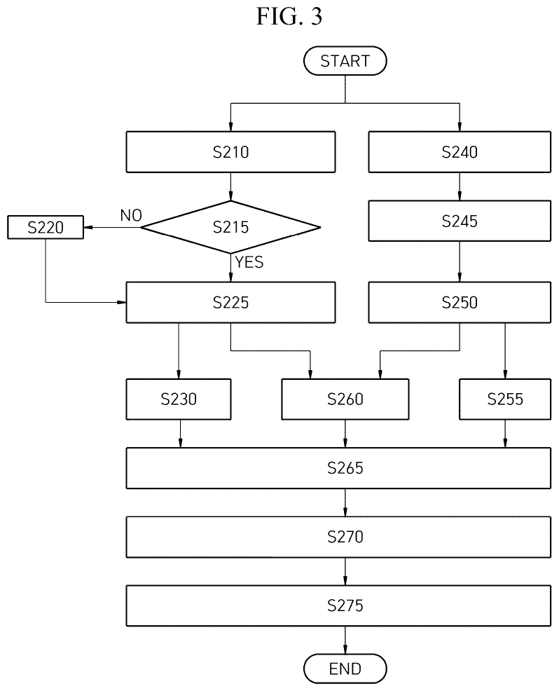

FIG. 1 is a block diagram illustrating a construction of an apparatus for predicting a degenerative brain function decline and a cognitive impairment according to an embodiment of the present disclosure. FIG. 2 is a diagram illustrating an example of input data and output data of the apparatus for predicting a degenerative brain function decline and a cognitive impairment.

As illustrated in FIG. 1, an apparatus 100 for predicting a degenerative brain function decline and a cognitive impairment (hereinafter abbreviated as a “prediction apparatus”) may be implemented in the form of a computer system.

The prediction apparatus 100 includes a processor 110, a communication device 120, memory 130, a storage device 140, an input interface device 150, an output interface device 160, and a bus 170. The prediction apparatus 100 illustrated in FIG. 1 is an embodiment. The components of the prediction apparatus 100 according to an embodiment of the present disclosure are not limited to the embodiment illustrated in FIG. 1, and a component may be added, changed, or deleted, if necessary.

The processor 110 may be a central processing unit (CPU) or may be a semiconductor device that executes a computer-readable instruction stored in the memory 130 or the storage device 140. The memory 130 and the storage device 140 may each include various types of volatile or non-volatile storage media. For example, the memory 130 may include read only memory (ROM) and random access memory (RAM). In an embodiment of the present specification, the memory 130 may be disposed inside or outside the processor 110 and connected to the processor 110 through various known means. The memory 130 includes various types of volatile or nonvolatile storage media, and may include ROM or RAM, for example.

Accordingly, an embodiment of the present disclosure may be implemented as a method implemented in a computer or may be implemented as a non-transitory computer-readable medium in which a computer-executable instruction has been stored. In an embodiment, when being executed by the processor 110, a computer-readable instruction may perform a method according to at least one aspect of this writing.

The communication device 120 may transmit or receive a wired signal or a wireless signal.

Furthermore, an operating method of the prediction apparatus 100 according to an embodiment of the present disclosure may be implemented in the form of a program instruction which may be executed through various computer means, and may be recorded on a computer-readable medium.

The computer-readable medium may include a program instruction, a data file, and a data structure alone or in combination. A program instruction recorded on the computer-readable medium may be specially designed and constructed for an embodiment of the present disclosure or may be known and available to those skilled in the computer software field. The computer-readable medium may include a hardware device configured to store and execute the program instruction. For example, the computer-readable medium may include magnetic media such as a hard disk, a floppy disk, and a magnetic tape, optical media such as CD-ROM and a DVD, magneto-optical media such as a floptical disk, ROM, RAM, and flash memory. The program instruction may include not only a machine code produced by a compiler, but a high-level language code capable of being executed by a computer through an interpreter.

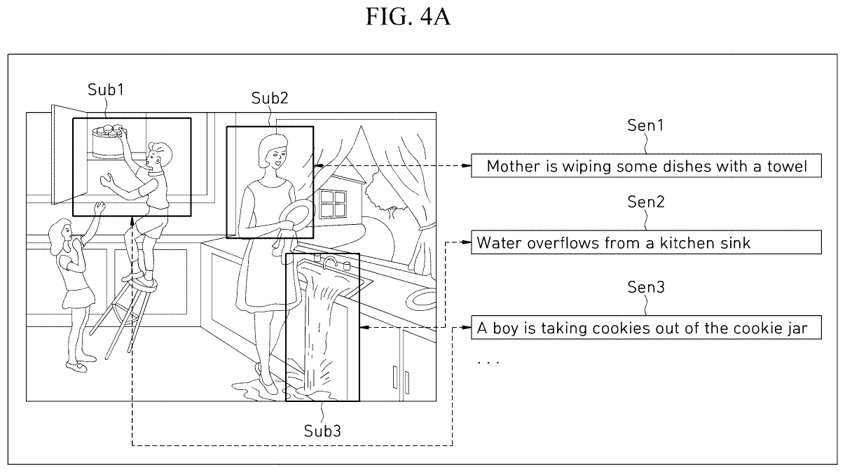

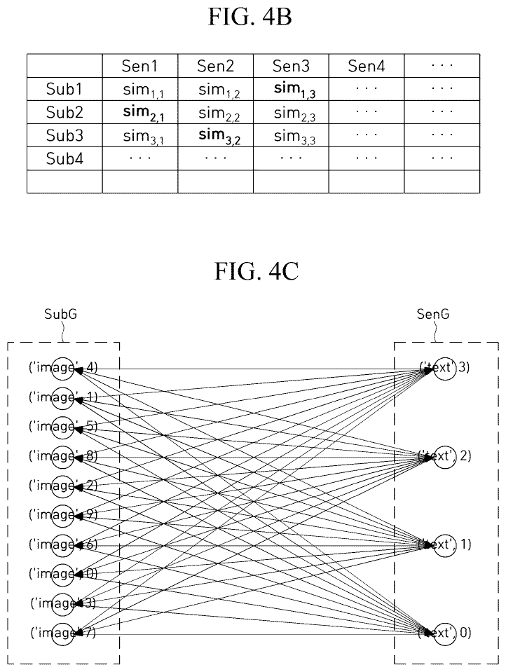

As described above, FIG. 2 is a diagram illustrating an example of input data and output data of the prediction apparatus 100. The prediction apparatus 100 receives an image D_{img} (hereinafter referred to as a “target image”) for a picture description task and utterance speech data D_{vc}, that is, speech data of a subject that are obtained in a process of the subject describing a target image. FIG. 4A illustrates an example of an image (target image) for a picture description task. The image (target image) that is used in the picture description task includes several situations. In the picture description task, a subject is required to describe several situations displayed in a target image. If the subject has a degenerative brain function decline or a cognitive impairment, the number of situations that is described by the subject, among the situations displayed in the target image will be small.

The fidelity of the contents of the description of a situation will be low although the subject describes the situation. For example, a subject of a normal group will describe the following level in the picture description task of the target image in FIG. 4A.

### [Example of Description of Normal Group Subject]

“The view of a kitchen can be seen. There are a mother, a son, and a daughter. The mother is wiping the water from a dish with a dishcloth in front of a sink. The son goes up on a stool, opens the lid of a cookie bowl in the top cabinet of the kitchen, and holds a cookie in his hand. The stool is about to fall. The mother is not aware of the situation. The daughter is reaching for cookies next to her brother. A sink is not turned off, so the water is overflowing the sink because the sink is full. The overflown water is falling toward the floor. There is a kitchen window over the sink, and curtains are fluttering in the wind. Outside the kitchen window, you can see a neighbor's house, a lawn is spread out in front of the neighbor's house, and you can see trees in the distance.”

However, a subject who has a degenerative brain function decline or cognitive impairment (hereinafter a “degenerative brain function decline or a cognitive impairment” is referred to as a “degenerative brain function decline”) will be low in the accuracy, fidelity, and richness of a description of each situation. Furthermore, according to research through eye tracking, it was found that a subject of a normal group describes the window, the curtain, and the situation outside the window, but a degenerative brain function decline patient rarely describes the window, the curtain, and the situation. That is, the degenerative brain function decline patient may never describe a specific scene within the target image.

The prediction apparatus 100 uses the utterance speech data D_{vc} that is obtained in a process of a subject describing a target image. The prediction apparatus 100 receives the target image D_{img} and the utterance speech data D_{vc} of the subject as an input, and classifies whether a subject is a subject who has a degenerative brain function decline or a subject of a normal group by using a vision language model (VLM) and a degenerative brain function decline and cognitive impairment prediction model based on a graph neural network. The prediction apparatus 100 outputs the results R_{cls} of the classification of the subject through the output interface device 160.

As a dataset related to a degenerative brain function decline, a dataset of an Alzheimer's disease (AD) is disclosed in DementiaBank (https://dementia.talkbank.org). Corpuses that are widely used, among datasets disclosed in DementiaBank, include Pitt, WLS, and Delaware. Furthermore, there are an ADReSS challenge dataset and an ADReSSo challenge dataset processed from the Pitt corpus. Many researches were carried out by using the two types (i.e., ADReSS and aDReSSo) challenge datasets. Performance of the prediction of a degenerative brain function decline and cognitive impairment model (hereinafter abbreviated as a “prediction model”) that is used by the prediction apparatus 100 according to an embodiment of the present disclosure may be validated through the several datasets. Furthermore, learning (training) that updates parameters of the prediction model may be performed by using the several datasets.

Furthermore, in addition to the disclosed benchmark datasets, the training and validation of the prediction model may also be performed through an utterance speech data file of a picture description task that is individually collected. All of datasets that are collected separately from the benchmark datasets include a speech file having a way format. There is a case in which utterance text, that is, the results of manual task transcription of a spoken speech, is provided. However, although transcription text is not present, a transcription text having sufficiently high quality may be obtained through an automatic transcription open-source library, such as Whisper of OpenAI.

The processor 110 included in the prediction apparatus 100 of FIG. 1 executes a method of predicting a degenerative brain function decline and a cognitive impairment, a method of displaying representative sentences of a normal group and a degenerative brain function decline group, a method of displaying keywords of a normal group and a degenerative brain function decline group, and a longitudinal analysis method for the early prediction of a degenerative brain function decline and a cognitive impairment, which will be described later, by executing computer-readable one or more instructions stored in the memory 130 or the storage device 140.

In the embodiment of FIG. 1, the one or more instructions may include an instruction to receive a target image of a picture description task and utterance speech data of a subject for the target image, an instruction to generate a sub-image embedding vector that is an embedding vector of a sub-image of the target image, an instruction to extract one or more sentences by segmenting utterance text that is generated by transcribing the utterance speech data in a sentence unit and to generate a sentence embedding vector that is an embedding vector of the sentence, and an instruction to calculate similarity between the sub-image and the sentence by using a vision language model and to determine that the subject corresponds to any one of a degenerative brain function decline and cognitive impairment group and a normal group based on the sub-image embedding vector, the sentence embedding vector, and the similarity.

The instruction to generate the sub-image embedding vector may include an instruction to generate the sub-image embedding vector by using the vision language model.

The instruction to generate the sentence embedding vector may include an instruction to generate the sentence embedding vector by using the vision language model.

The instruction to determine that the subject corresponds to any one of the degenerative brain function decline and cognitive impairment group and the normal group may include an instruction to calculate the similarity between the sub-image and the sentence by using the vision language model, an instruction to generate a bipartite graph including a sub-image node corresponding to the sub-image embedding vector and a sentence node corresponding to the sentence embedding vector, wherein a weight of an edge that connects the sub-image node and the sentence node is set in the bipartite graph based on the similarity, an instruction to input the bipartite graph to a graph neural network and to generate a graph-level embedding vector through information propagation, and an instruction to calculate a probability that the subject is to belong to the normal group by inputting the graph-level embedding vector to a classifier based on a pre-trained artificial neural network and to classify the subject as any one group of the degenerative brain function decline and cognitive impairment group and the normal group based on the probability.

The graph neural network may be a graph convolution neural (GCN) network.

The one or more instructions may further include an instruction to extract a speech feature of the subject from the utterance speech data and an instruction to generate a speech embedding vector based on the speech feature. In this case, the instruction to classify the subject may include an instruction to calculate the probability by inputting the graph-level embedding vector and the speech embedding vector to the classifier.

The one or more instructions may further include an instruction to extract a text feature by inputting the utterance text to a language model and an instruction to generate the text embedding vector based on the text feature. In this case, the instruction to classify the subject may include an instruction to calculate the probability by inputting the graph-level embedding vector and the text embedding vector to the classifier.

The one or more instructions may further include an instruction to generate a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed, an instruction to calculate first similarity between the first representative embedding vector and the sentence embedding vector of the degenerative brain function decline and cognitive impairment group and to generate a similarity high-rank sentence group by grouping sentence embedding vectors corresponding to a predetermined high-rank percentage, among all of the sentence embedding vectors of the degenerative brain function decline and cognitive impairment group, based on the first similarity, an instruction to calculate second similarity between the second representative embedding vector and the sentence embedding vector of the degenerative brain function decline and cognitive impairment group and generating a similarity low-rank sentence group by grouping sentence embedding vectors corresponding to a predetermined low-rank percentage, among all of the sentence embedding vectors of the degenerative brain function decline and cognitive impairment group, based on the second similarity, and an instruction to select representative sentences corresponding to the similarity high-rank sentence group and the similarity low-rank sentence group, respectively, based on a predetermined reference and displaying the representative sentences through an output interface device.

The one or more instructions may further include an instruction to generate a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed, an instruction to calculate first similarity between the first representative embedding vector and the sentence embedding vector of the degenerative brain function decline and cognitive impairment group, to generate a first relevant sentence set by grouping sentence embedding vectors corresponding to a predetermined high-rank percentage, among all of the sentence embedding vectors of the degenerative brain function decline and cognitive impairment group, based on the first similarity, and to generate a first irrelevant sentence set by grouping sentence embedding vectors corresponding to a predetermined low-rank percentage, among all of the sentence embedding vectors of the degenerative brain function decline and cognitive impairment group, based on the first similarity, an instruction to calculate second similarity between the second representative embedding vector and the sentence embedding vector of the degenerative brain function decline and cognitive impairment group, to generate a second relevant sentence set by grouping sentence embedding vectors corresponding to a predetermined low-rank percentage, among all of the sentence embedding vectors of the degenerative brain function decline and cognitive impairment group, based on the second similarity, and to generate a second irrelevant sentence set by grouping sentence embedding vectors corresponding to a predetermined high-rank percentage, among all of the sentence embedding vectors of the degenerative brain function decline and cognitive impairment group, based on the second similarity, an instruction to calculate third similarity between the second representative embedding vector and the sentence embedding vector of the normal group, to generate a third relevant sentence set by grouping sentence embedding vectors corresponding to a predetermined high-rank percentage, among all of the sentence embedding vectors of the normal group, based on the third similarity, and to generate a third irrelevant sentence set by grouping sentence embedding vectors corresponding to a predetermined low-rank percentage, among all of the sentence embedding vectors of the normal group, based on the third similarity, an instruction to calculate fourth similarity between the first representative embedding vector and the sentence embedding vector of the normal group, to generate a fourth relevant sentence set by grouping sentence embedding vectors corresponding to a predetermined low-rank percentage, among all of the sentence embedding vectors of the normal group, based on the fourth similarity, and to generate a fourth irrelevant sentence set by grouping sentence embedding vectors corresponding to the predetermined high-rank percentage, among all of the sentence embedding vectors of the normal group, based on the fourth similarity, an instruction to select a word not included in the third relevant sentence set, among words included in the first relevant sentence set, and to set the selected word as a first keyword, to select a word not included in the fourth relevant sentence set, among words included in the second relevant sentence set, and to set the selected word as a second keyword, to select a word not included in the third irrelevant sentence set, among words included in the first irrelevant sentence set, and to set the selected word as a third keyword, to select a word not included in the fourth irrelevant sentence set, among words included in the second irrelevant sentence set, and to set the selected word as a fourth keyword, and an instruction to output the first keyword and the second keyword as keyword that help in determining the degenerative brain function decline and cognitive impairment group and to output the third keyword and the fourth keyword as keywords that do not help in determining the degenerative brain function decline and cognitive impairment group.

The one or more instructions may further include an instruction to calculate the probability for an identical subject with respect to different target images at regular time intervals during a predetermined period by a predetermined number of times and an instruction to input the probability calculated during the predetermined period to a pre-trained longitudinal analysis model and to determine that the subject is to belong to the degenerative brain function decline and cognitive impairment group after a predetermined period based on an output of the longitudinal analysis model.

FIG. 3 is a flowchart for describing a method of predicting a degenerative brain function decline and a cognitive impairment (hereinafter abbreviated as a “prediction method”) according to a first embodiment of the present disclosure. The prediction method may be performed by the prediction apparatus 100. An embodiment in which the prediction apparatus 100 performs each of steps included in the prediction method, for convenience's sake, is described.

Referring to FIG. 3, the prediction method according to a first embodiment of the present disclosure includes steps S210 to S275. The prediction method illustrated in FIG. 3 is based on an embodiment. The steps of the prediction method according to an embodiment of the present disclosure are not limited to the embodiment illustrated in FIG. 3, and a step may be added, changed, or deleted, if necessary.

Step S210 is a step of receiving a target image of a picture description task. Step S215 is a step of determining whether the target image is a color image. Step S220 is a step (coloring step) of converting the target image into a color image.

The prediction apparatus 100 receives the target image of the picture description task through the communication device 120 or the input interface device 150 (S210). When the target image is not a color image (S215), that is, the target image is a white and black image or a grayscale image, the processor 110 converts the target image into a color image (S220). The processor 110 may color the target image through a generation model (or a generative model). As another example, the coloring of the target image may be performed through a manual task. Why the target image is converted into the color image is that a vision language model (VLM) cannot properly process a white and black image.

Step S225 is a step of generating (image cropping step) a sub-image of the target image through image cropping.

When the target image is a color image or after the target image is converted into a color image, the processor 110 generates one or more sub-images by cropping the target image in a situation unit that appears in the target image. For reference, the “cropping” is a task of cutting a part of an image.

For example, the processor 110 may generate a first sub-image by cutting only a part in which a situation where the son takes out a cookie appears by centering on the part in the picture (target image) of FIG. 4A, may generate a sub-image by cutting only a part in which a situation where the stool is about to fall appears by centering on the situation, may generate a sub-image by cutting only a part in which a situation where the mother is wiping the water from the dish with the dishcloth appears by centering on the situation, and may generate a sub-image by cutting only a part in which a situation where the water is overflowing the sink by centering on the situation. The processor 110 performs such an image cropping task on a set number N of sub-images. According to the setting, the sub-image generated by the processor 110 may include an image (not-cropped image) having a total size for describing an overall situation.

Step S230 is a step of generating a sub-image embedding vector by using a vision language model (VLM).

The processor 110 performs sub-image embedding by using the VLM. That is, the processor 110 generates the sub-image embedding vector, that is, an embedding vector of the sub-image of the target image, by using the VLM. The VLM has been described at the beginning of the detailed description in detail.

The embedding task of the sub-image that is performed in this step is a task of converting each pixel value of the sub-image into a vector of computer-readable digitized M dimensions. M may be given as various dimensions, such as a 256 dimension or a 768 dimension. In the embedding task of the processor 110, the VLM may not be used (common image embedding is also possible), but in an embodiment of the present disclosure, the VLM is used in order to improve performance in determining and predicting a degenerative brain function decline.

As a detailed example, the processor 110 may obtain an embedding vector corresponding to an sentence that is extracted based on utterance speech data of a subject as a pair with respect to each sub-image through the VLM, and may obtain the embedding vector of a corresponding sub-image by averaging the values of the embedding vectors of the sub-image for all of the sentences.

A vision language model (VLM) follows a method of simultaneously learning an image and text, that is, expressions of two modalities. That is, the processor 110 extracts an image expression into which text context has been incorporated when expressing the embedding vector of the image by using the VLM, and likewise extracts a text expression into which image context has been incorporated when extracting the embedding vector of the text.

Assuming that n sub-images and m sentences are present, when the processor 110 extracts the embedding vector of the first sub-image by using VLM, the VLM determines the image embedding of the first sub-image from the viewpoint of each of pieces of text from the first sentence to an m-th sentence. Likewise, when the processor 110 extracts the image embedding vector of the second sub-image, the VLM determines the embedding of the second sub-image from the viewpoint of each of pieces of text from the first sentence to the m-th sentence. Furthermore, when the processor 110 extracts the text embedding vector of the first sentence by using the VLM, the VLM determines the text embedding of the first sentence from the viewpoint of an image from the first image to an n-th image.

Accordingly, in order to extract a final sub-image embedding vector for the first sub-image, the processor 110 integrates m image embedding vectors for the first sub-image. For example, the processor 110 may determine the mean of the m image embedding vectors for the first sub-image as the embedding vector (or sub-image embedding vector) of the first sub-image. Furthermore, the processor 110 may determine the mean of n text embedding vectors for the first sentence as the embedding vector (or sentence embedding vector) of the first sentence.

Among the aforementioned contents, a portion in which the sub-image embedding vector is generated corresponds to step S225, and a portion in which the sentence embedding vector is generated corresponds to step S255.

Step S240 is a step of receiving utterance speech data of a subject. Step S245 is a step of generating utterance text through automatic transcription.

The prediction apparatus 100 receives the utterance speech data of the subject that describes the target image through the communication device 120 or the input interface device 150 (S240). The utterance speech data may be given in the form of a way file.

Furthermore, the processor 110 generates the utterance text by transcribing the utterance speech data (S245). For example, the processor 110 converts the utterance speech data into utterance text through an automatic transcription open-source library (e.g., Whisper of OpenAI).

Step S250 is a step of segmenting the utterance text in a sentence unit. Step S255 is a step of generating a sentence embedding vector by using the VLM.

The processor 110 extracts one or more sentences (S250) by segmenting the utterance text that is generated by transcribing the utterance speech data in a sentence unit, and generates the sentence embedding vector that is, an embedding vector of the sentence, by using the VLM (S255).

In step S250, the processor 110 extracts one or more sentences from the utterance text by segmenting the utterance text based on sentential marks, such as a period, an exclamation mark, and a question mark. When the length of a sentence is greater than a critical value, the sentence may be segmented by a comma.

Thereafter, in step S255, the processor 110 performs an embedding task on each sentence that is generated as the results of the segmentation by using the VLM. One of the functions of the VLM is to generate a proper caption of a given image. The VLM includes a function of extracting a feature of a sentence therein. The processor 110 may obtain the embedding vector of each sentence through the feature extraction function of the VLM. In this case, the processor 110 sets the dimension of the embedding vector to be identical with the dimension of the sub-image embedding vector. The embedding vector dimension M may be a 256 dimension or a 768 dimension. The embedding task may not use the VLM (common sentence embedding is also possible), but in an embodiment of the present disclosure, the VLM is used to improve prediction performance of a degenerative brain function decline and a cognitive impairment.

As a detailed example, the processor 110 may obtain embedding vectors corresponding to pairs of all of sub-images with respect to each sentence through the VLM, and may obtain the embedding vector of a corresponding sentence by averaging the values of the embedding vector of the sentence for all of the sub-images.

Detailed contents of the method of generating the sentence embedding vector has been described along with the method of generating the sub-image embedding vector in step S230.

Step S260 is a step of calculating similarity between the sub-image and the sentence by using the VLM.

In this step, the processor 110 calculates similarity between all of the sub-images and all of the sentences by using the VLM. Step S260 may be performed by using the VLM. An image-text matching function, among the functions of the VLM, is used. The processor 110 may calculate cosine similarity between the sub-image and the sentence through the image-text matching function. That is, the processor 110 calculates the cosine similarity with respect to all of pairs of the sub-images and the sentences. For example, in step S260, in order to enhance multi-modal alignment (to make information having the same meaning have a similar expression in different modes), a process of scaling information in a trained log scale after L2 normalization by using an ITC head provided by a library for language-vision intelligence (LAVIS) may be added.

Step S260 is performed separately from steps S230 and S255. When steps S225 and S250 are completed, the processor 110 may perform step S260.

Steps S265 to S275 are steps of determining that the subject corresponds to any one of a degenerative brain function decline and cognitive impairment group and a normal group based on the similarity calculated in step S260.

Step S265 is a step of generating a bipartite graph including a sub-image node and a sentence node.

In this step, the processor 110 generates the bipartite graph including a sub-image node corresponding to the sub-image embedding vector and a sentence node corresponding to the sentence embedding vector. In this case, the processor 110 sets the weight of an edge that connects the sub-image node and the sentence node in the bipartite graph based on the similarity between the sub-image and the sentence.

As a detailed example, the sub-image embedding vector, the sentence embedding vector, and the cosine similarity between the sub-image and the sentence become the sub-image node and sentence node of the bipartite graph and the weight of the edge, respectively.

For reference, the graph includes a node and an edge. The edge connects two nodes. The bipartite graph includes two types of nodes. An edge in the bipartite graph is possible only between nodes having different types. If a connection between the same types is present, a graph is not a bipartite graph.

A node included in a bipartite graph according to an embodiment of the present disclosure includes only two types of sub-image node and text node. Only a connection between an image and text is present. Each embedding vector is set as the attribute of a node. Cosine similarity is set as the weight of an edge.

The bipartite graph generated in step S265 includes all of pieces of relation information between the sub-image and the sentence. The bipartite graph captures complex relation information between contents of the subject, which are described with an utterance, while watching the target image, and the sub-image. First, when subjects describe parts of a target image, a sentence related to a specific sub-image has a great edge weight. Second, if a participant provides a description that covers all of situations because a full description of all of situations within the target image is important, a sub-image nod is connected to a related sentence node and has at least one great edge weight. Third, an utterance not related to the target image does not have a great edge weight even with any image node. The bipartite graph according to an embodiment of the present disclosure includes various types of implicit information, including the aforementioned aspects. If the various types of implicit information including the aforementioned aspects are properly used, prediction performance of a degenerative brain function decline can be greatly improved.

FIGS. 4A to 4C are diagrams for describing a process of generating a bipartite graph indicative of a relation between an image and text.

Contents related to step S265 are illustrated in FIGS. 4A to 4C. As illustrated in FIG. 4A, the processor 110 may extract one or more sub-images in the target image through image cropping. For example, the processor 110 may extract a sub-image (Sub1) regarding the situation where the son takes out a cookie, a sub-image (Sub2) regarding the situation where the mother is wiping the water from the dish with the dishcloth, and a sub-image (Sub3) regarding the situation where the water is overflowing the sink. Furthermore, sentences Sen1, Sen2, and Sen3 extracted from the utterance text have different similarities with respect to the sub-images, respectively.

As described above, the processor 110 extracts the sub-image from the target image in order to capture a relation between the sub-image and the sentence, segments the utterance speech data of the subject in a sentence unit by transcribing the utterance speech data in text, and then calculates the cosine similarity between all of the sub-images and the sentence pairs by using the VLM. The processor 110 may display the calculated similarities as in a similarity matrix of FIG. 4B. That is, the similarity matrix is a matrix indicative of similarities between the sub-images Sub1, Sub2, and Sub3 and the sentences Sen1, Sen2, and Sen3, and may be said to be relation information between the sub-image and the sentence.

The similarity matrix itself may become an adjacency matrix having edge weights as elements because the cosine similarity of the similarity matrix has a value of 0 to 1. The similarity matrix of FIG. 4B may be expressed in the form of a bipartite graph of FIG. 4C because the adjacent matrix may be expressed in the form of a graph. The bipartite graph includes sub-image nodes (SubG) corresponding to sub-images, respectively, and sentence nodes (SenG) corresponding to sentences, respectively. The similarity of the similarity matrix is applied to the weight of an edge that connects the sub-image node and the sentence node.

Step S270 is a step of generating a graph-level embedding vector by using a graph neural network (GNN). Step S275 is a step of classifying the subject as a degenerative brain function decline group or a normal group.

The processor 110 inputs the bipartite graph generated in step S265 to the GNN and generates the graph-level embedding vector through information propagation (S270). The GNN may be a graph convolution neural network. In this case, a GNN which may be applied in an embodiment of the present disclosure is not limited to the GNN.

The bipartite graph generated in step S265 is given as an input to the GNN. The GNN has been described in detail at the beginning of the detailed description. In the present embodiment, in particular, a graph convolution neural network (GCN), among various graph neural networks, is used. Several layers are possible for a GCN layer. For example, three GCN layers may be set. According to experiments, it was found that performance is the highest when three GCN layers are used with respect to most of data. In this case, that is, an optimal number of GCN layers may be different depending on the type of data or the number of samples. Accordingly, in an embodiment of the present disclosure, the number of GCN layers is not limited. In the GCN, the first adjacent information is collected because information is propagated through one edge if the information passes through one GCN layer. Up to the second adjacent information is collected because the information is propagated through two steps of edges if the information passes through two GCN layers. After the information passes through each GCN layer, efficiency and performance of neural network learning can be improved by applying batch normalization.

The processor 110 sets the initial embedding of the sub-image node included in the bipartite graph as the sub-image embedding vector, and sets the initial embedding of the sentence node as the sentence embedding vector. The sub-image embedding vector and the sentence embedding vector are propagated to neighbors when passing through the GCN layer. The processor 110 sets a corresponding propagated weight so that the weight is proportional to the cosine similarity between the sub-image and the sentence. That is, information propagation is performed more greatly between a sub-image and a sentence having high similarity. A final embedding vector (final node embedding vector) of each node included in the bipartite graph may be obtained through several GCN layers.

In order to classify a subject as any one group of a degenerative brain function decline group and a normal group, it is necessary to determine whether a bipartite graph corresponding to a subject corresponds to the degenerative brain function decline group or the normal group. To this end, an embedding vector needs to be obtained at a graph level.

That is, the last process of learning through the GNN is a task for obtaining a graph-level embedding vector based on the embedding vectors of all of the nodes of the bipartite graph (S270) and classifying the subject as any one of a degenerative brain function decline group AD or MCI and a normal group HC by passing graph-level embeddings through a pre-trained artificial neural network (e.g., a linear layer) (S275).

Hereinafter, a process of the processor 110 obtaining a graph level embedding vector is described.

A method of collecting, by the processor 110, embedding information of the nodes of the bipartite graph may be obtained by using all of simple methods, such as a global mean pooling method of calculating the mean of the embeddings of nodes and a global max pooling method using only a maximum value, among the embeddings of nodes, and a complicated method, such as hierarchical approaches such as DiffPool and gPool.

A case in which the processor 110 calculates a graph-level embedding vector by using the global mean pooling method is described below. Assuming that graph-level embedding trained through the GCN with respect to a subject (sample) “s” is hₛ, a corresponding value is calculated as in Equation 2.

$${h}_{s} = \frac{1}{2} ( 〈 {h}_{s , i}^{v} 〉 + 〈 {h}_{s , j}^{t} 〉 ) \qquad ( 2 )$$

In Equation 2, [symbol] denotes an average operation. h_{s,i}^{v} and h_{s,j}^{t} denote final node embedding vectors of a sub-image node and a sentence node (hereinafter referred to as a “final node embedding vector”), respectively. That is, the final node embedding vector (including a final sub-image node embedding vector and a final sentence node embedding vector) is a node embedding vector trained through a graph neural network (GNN). According to Equation 2, the mean of node embedding vectors may be calculated at a ratio equal to the ratio of sub-image nodes (1:1) regardless of the number of sentence nodes.

After the graph-level embedding vector hₛ is obtained through Equation 2, in step S275, the processor 110 calculates the probability (y) (normal probability) that the subject will belong to the normal group by inputting the graph-level embedding vector to a classifier based on a pre-trained artificial neural network (e.g., a linear layer) and classifies the subject as any one group of the degenerative brain function decline group and the normal group based on the probability (y). An equation for calculating the normal probability (y) may be expressed as in Equation 3.

$$y = Linear ( {h}_{s} ) \qquad ( 3 )$$

hₛ, that is, the graph-level embedding vector, may have various dimension values (high dimension such as a 256 dimension) depending on a GCN structure. The role of the linear layer is to convert the graph-level embedding vector so that the graph-level embedding vector has a 1-dimension scalar value. For reference, in the classifier (linear conversion model), a weight between variables may be trained through learning data (i.e., D_{img}, D_{vc}, and R_{cls} in FIG. 2) of the picture description task. The probability (y), that is, a final result value, has a value within a range of 0 to 1. The subject has a high probability “1-y” that the subject will correspond to the degenerative brain function decline group AD or MCI as the probability (y) is closer to 0, and has a high probability (y) that the subject will correspond to the normal group HC as the probability (y) is closer to 1.

The processor 110 optimizes parameters (e.g., W₁¹ and W₂¹ in Equation 1) of the graph neural network by calculating a difference between the normal probability (y) calculated through the classifier and a label (i.e., the results of the classification) through cross entropy and calculating a loss thereof so that a validation loss is minimized through back propagation.

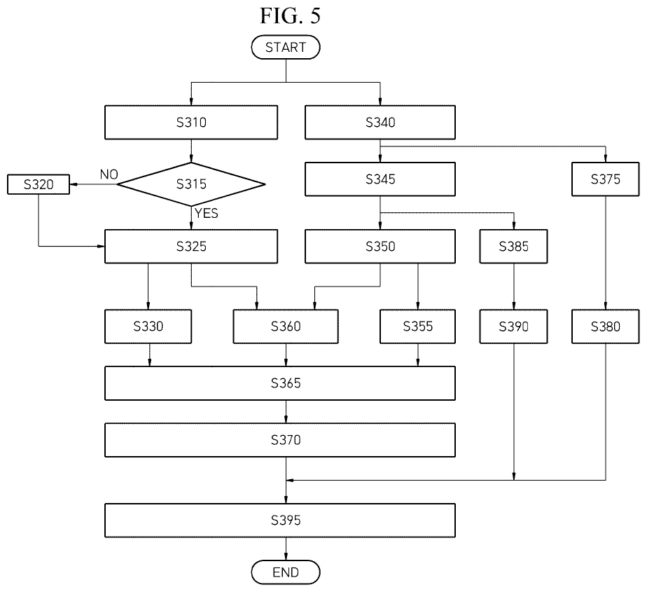

FIG. 5 is a flowchart for describing a method of predicting a degenerative brain function decline and a cognitive impairment (hereinafter abbreviated as a “prediction method”) according to a second embodiment of the present disclosure. As in the first embodiment, the prediction method of FIG. 5 may be performed by the prediction apparatus 100.

Referring to FIG. 5, the prediction method according to a second embodiment of the present disclosure includes steps S310 to S395. The prediction method illustrated in FIG. 5 is based on an embodiment. The steps of the prediction method according to an embodiment of the present disclosure are not limited to the embodiment of FIG. 5, and a component may be added, changed, or deleted, if necessary.

As illustrated in FIG. 5, in the second embodiment, steps S375, S380, S385, and S390 are added to the first embodiment (refer to FIG. 3), and a text embedding vector S390 and a speech embedding vector S380 are added so that data input to the classifier in step S395 are changed. That is, the second embodiment is an embodiment in which speech feature information and utterance text feature information of a subject are additionally used compared to the first embodiment and is intended to improve degenerative brain function decline determine/prediction performance. In other words, if the relation between the image and the sentence has been used through the VLM in the first embodiment of FIG. 3, the second embodiment of FIG. 5 can improve prediction performance of a degenerative brain function decline and a cognitive impairment in addition to information on a speech itself and text itself.

Hereinafter, the embodiment of FIG. 5 is described in detail. Steps S310 to S370 correspond to the steps of the first embodiment, respectively, and are the same as the steps of the first embodiment. That is, step S310 is identical with step S210. Step S315 is identical with step S215. Step S320 is identical with step S220. Step S325 is identical with step S225. Step S330 is identical with step S230. Step S340 is identical with step S240. Step S345 is identical with step S245. Step S350 is identical with step S250. Step S355 is identical with step S255. Step S360 is identical with step S260. Step S365 is identical with step S265. Step S370 is identical with step S270. Only steps S375, S380, S385, S390, and S395 are described because other steps are the same as those of the first embodiment.

Step S375 is a step of extracting the speech feature of the subject from the utterance speech data. This step is performed after the utterance speech data input step S340.

The processor 110 uses a library that extracts a speech signal itself as an acoustic embedding vector as in wav2vec2.0 in this process. Specifically, the processor 110 segments the utterance speech data (speech file) in a predetermined unit, and obtains the acoustic embedding vector, that is, a speech feature, with respect to each segmented speech interval. The unit of segmentation may have an optimal unit for each dataset. For example, the unit of segmentation may be set to 20 ms.

Step S380 is a step of generating a speech embedding vector by using a learning model based on the speech feature.

The processor 110 may generate the speech embedding vector by inputting the acoustic embedding vector, that is, the speech feature of the subject, to a transformer that is a learning model.

Step S385 is a step of extracting the text feature of the utterance text by using a language model. This step is performed after an utterance text generation step S345.

The processor 110 may use a pre-trained language model in the text feature extraction step. The language model may include several types of models. For example, the processor 110 may use bidirectional encoder representations from transformers (BERT) in extracting the text feature of a subject. In this case, the processor 110 obtains a text feature vector (may be denoted as a “text feature”) of a 768 dimension as the results of an input by inputting utterance text to the BERT, that is, a language model, as a token. If text is too long, the text cannot be input to the BERT. Accordingly, when utterance text is longer than a 512 token that is an input critical value, the processor 110 segments the utterance text and then obtains a text feature vector by inputting the segmented utterance text to the BERT.

Step S390 is a step of generating a text embedding vector by inputting the text feature to the learning model.

Furthermore, the processor 110 generates a text embedding vector by inputting the text embedding vector to a transformer. In step S390, it is preferred that a separate transformer having a parameter different from that of the transformer used in step S380 is used.

Step S395 is a step of classifying the subject as a degenerative brain function decline group or a normal group.

In this step, the processor 110 calculates the probability that the subject will belong to the normal group (the normal probability (y)) by inputting at least any one of the graph-level embedding vector, the speech embedding vector, and the text embedding vector or a combination of them to the classifier based on a pre-trained artificial neural network, and classifies the subject as the degenerative brain function decline group or the normal group based on the normal probability (y).

For example, the processor 110 may calculate the normal probability (y) by inputting the graph-level embedding vector, the speech embedding vector, and the text embedding vector to the classifier. As another example, the processor 110 may calculate the normal probability (y) by inputting the graph-level embedding vector and the speech embedding vector to the classifier. As still another example, the processor 110 may calculate the normal probability (y) by inputting the graph-level embedding vector and the text embedding vector to the classifier.

If the subject is classified by inputting the graph-level embedding vector, the speech embedding vector, and the text embedding vector to the classifier, the processor 110 may generate a final embedding vector (“final embedding vector”) by concatenating the graph-level embedding vector, the speech embedding vector, and the text embedding vector or merging the graph-level embedding vector, the speech embedding vector, and the text embedding vector in various ways. In step S395, the processor 110 calculates the probability (y) that the subject will belong to the normal group by inputting the final embedding vector to the classifier based on a pre-trained artificial neural network (e.g., a linear layer) instead of the graph-level embedding vector in Equation 3. A subsequent process is the same as step S275, and thus a detailed description thereof is omitted.

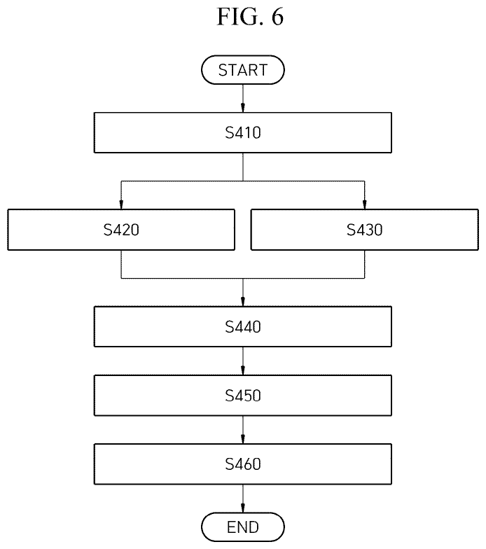

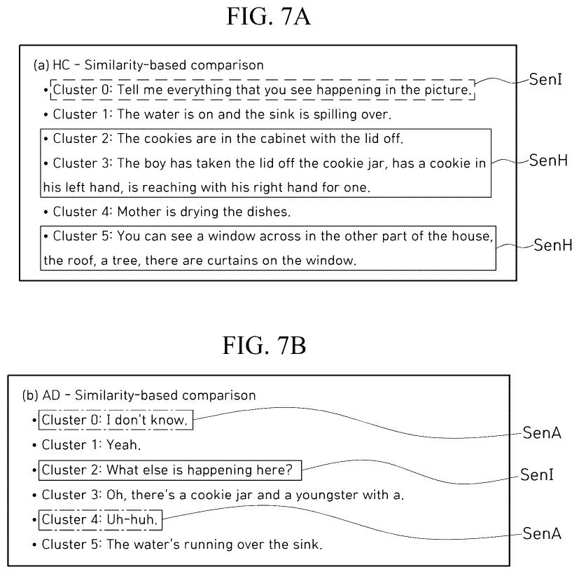

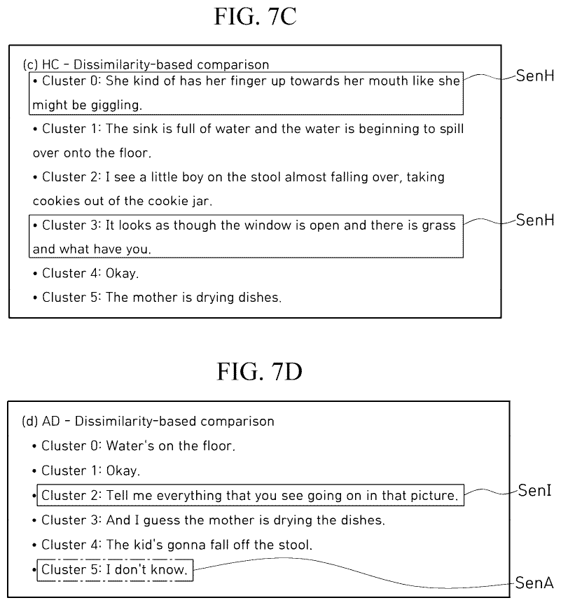

FIG. 6 is a flowchart for describing a method of displaying representative sentences of a normal group and a degenerative brain function decline group (hereinafter abbreviated as a “representative sentence display method”) through an explainable model for the prediction of a degenerative brain function decline and a cognitive impairment (hereinafter abbreviated as a “representative sentence display method”) according to an embodiment of the present disclosure. Furthermore, FIGS. 7A to 7D are diagrams illustrating examples of representative sentences of a normal group and a degenerative brain function decline group, which are derived through a similarity-based comparison and a dissimilarity-based comparison.

The representative sentence display method of FIG. 6 is a method of describing a difference between representative sentences of a normal group and a degenerative brain function decline group through the explainable model for the prediction of a degenerative brain function decline and a cognitive impairment.

The representative sentence display method may be performed by the prediction apparatus 100. An embodiment in which the prediction apparatus 100 performs steps included in the representative sentence display method, for convenience's sake, is described.

Referring to FIG. 6, the representative sentence display method according to an embodiment of the present disclosure includes steps S410 to S460. The representative sentence display method illustrated in FIG. 6 is based on an embodiment. The steps of the representative sentence display method according to an embodiment of the present disclosure are not limited to the embodiment illustrated in FIG. 6, and a component may be added, changed, or deleted, if necessary.

The representative sentence display method of FIG. 6 is executed after the execution of the prediction method of FIG. 3 or 5. Before the representative sentence display method of FIG. 6 is executed, the processor 110 trains a prediction model (the graph neural network and the classifier) by using the learning data (D_{img}, D_{vc}, and R_{cls} in FIG. 2) of the picture description task according to the embodiment of FIG. 3 or 5, and extracts a learning data sample that accords with an answer based on the prediction model (i.e., a model corresponding to the best epoch) having the best prediction performance and the results of the classification of the classifier. The representative sentence display method of FIG. 6 is performed based on a learning data sample that accords with an answer on the basis of a prediction model indicative of the best prediction performance.

Step S410 is a step of generating representative embedding vectors of a degenerative brain function decline group and a normal group.

The processor 110 extracts a representative embedding vector (h_{AD}) of the degenerative brain function decline group and a representative embedding vector (h_{HC}) of the normal group based on the results of the classification of the prediction model for learning data. The representative embedding vector of each group (each of the degenerative brain function decline group and the normal group, hereinafter the same) is obtained by averaging all of trained node embedding vectors (final node embedding vectors) that accord with an answer in a corresponding group. For example, the processor 110 obtains the representative embedding vector of the degenerative brain function decline group by averaging final node embedding vectors when learning data classified as the degenerative brain function decline group are identical with an answer (label). Any one of a final sentence node embedding vector and a final sub-image node embedding vector or a combination of them may be used as the final node embedding vector. For example, the processor 110 may obtain the representative embedding vector by calculating the mean of final sentence node embedding vectors on which the training of a graph neural network has been completed, and may obtain the representative embedding vector by calculating the mean of a final sentence node embedding vector and a final sub-image node embedding vector on which the training of a graph neural network has been completed.

A process subsequent to step S410 is performed by dividing the process into step S420 and step S430.

Step S420 is a similarity-based comparison step and is a step of extracting a similarity high-rank sentence for the representative embedding vector of each group.

For example, the processor 110 extracts a sentence up to a cosine similarity high-rank 20% by comparing the representative embedding vector of the same group and the embedding vector of each sentence with respect to each group. A set of such sentences (i.e., from a sentence having the highest similarity to a sentence up to a high-rank 20% in descending power) is denoted as each of S_{AD}, and S_{HC, ˜} with respect to each of the degenerative brain function decline group and the normal group. That is, S_{AD}, is a set of sentences corresponding to the representative embedding vector of the degenerative brain function decline group and the sentence embedding vector of the degenerative brain function decline group having similarity of a critical value or more. In other words, S_{AD, ˜} may be said to be a similarity high-rank sentence group of the degenerative brain function decline group for the representative embedding vector of the degenerative brain function decline group. Likewise, S_{HC, ˜} is the similarity high-rank sentence group of the normal group for the representative embedding vector of the normal group.

When the embedding vector of a specific sentence has high similarity with the representative embedding vector of the same group, this means that the prediction model importantly uses the specific sentence in classifying a subject as the degenerative brain function decline group or the normal group. Accordingly, the representative sentence of the degenerative brain function decline group, which is derived from the picture description task for a specific target image, becomes an important clue in the research of a user (e.g., an investigator).

Step S430 is a dissimilarity-based comparison step, and is a step of extracting a similarity low-rank sentence for the representative embedding vector of a counterpart group of each group.

For example, the processor 110 extracts the sentence up to the cosine similarity low-rank 20% by comparing the representative embedding vector of a counterpart group (i.e., a counterpart group of the degenerative brain function decline group is the normal group, and a counterpart group of the normal group is the degenerative brain function decline group) for each group and the embedding vector of each sentence. A set of such sentences (i.e., from a sentence having the lowest similarity to a sentence up to a low-rank 20% in ascending power) is denoted as each of [symbol] and [symbol] with respect to each of the degenerative brain function decline group and the normal group. [symbol] is the similarity low-rank sentence group of the degenerative brain function decline group for the representative embedding vector of the normal group. [symbol] is the similarity low-rank sentence group of the normal group for the representative embedding vector of the degenerative brain function decline group. Through step S430, a sentence that is used differentially from a counterpart group may be selected among the sentences of each group.

Step S440 is a step of obtaining the text embedding vector of the extracted sentence.

The processor 110 extracts the sentence embedding vectors of sentences that belong to the sentence groups S_{AD, ˜}, S_{HC, ˜}, [symbol] , and [symbol] obtained through steps S420 and S430.

The processor 110 may use a pre-generated sentence embedding vector, but may use an open-source library that converts a sentence into an embedding vector, such as a sentence transformer, because an image is not separately present and only a sentence is present. If the sentence transformer is used, text embedding may be extracted independently of a sub-image. That is, it is not necessary to match the sub-image.

Step S450 is a step of grouping the extracted sentence through clustering.

For example, the processor 110 performs grouping on each of the sentence groups S_{AD, ˜}, S_{HC, ˜}, [symbol] , and [symbol] by using a k-means clustering scheme. The number of clusters may be set to a specific number (e.g., 5). In this case, in an embodiment of the present disclosure, the number of clusters is not limited. The processor 110 may group similar sentences within the same sentence group through clustering.

Step S460 is a step of displaying the representative sentence of each of the clusters of the degenerative brain function decline group and the normal group.

When the clustering of each sentence group is completed through step S450, the processor 110 extracts a sentence (i.e., the representative sentence of each cluster) that is closest to each of the centers of one or more clusters, which are included in each sentence group, and transmits the sentence to the terminal of a user (e.g., an investigator) through the communication device 120 or the output interface device 160 or outputs the sentence so that a user can check the sentence.

FIGS. 7A to 7D are diagrams illustrating examples of the representative sentences of clusters included in each sentence group. FIG. 7A is the representative sentences of clusters included in the sentence group S_{HC, ˜}. FIG. 7B is the representative sentences of clusters included in the sentence group S_{AD, ˜}. FIG. 7C is the representative sentences of clusters included in the sentence group [symbol] . FIG. 7D is the representative sentences of clusters included in the sentence group [symbol] .

In the datasets of FIGS. 7A to 7D, SenI denotes the utterance of an investigator, SenH denotes a sentence (e.g., a detailed description) that well indicates the characteristics of the normal group HC. SenA denotes a sentence (e.g., an answer that I do not know or filler words, such as “Uh-huh”) that well indicates the characteristics of the degenerative brain function decline group AD.

The prediction apparatus 100 may give insight into a characteristic sentence of each of the degenerative brain function decline group and the normal group to an investigator by displaying the representative sentence of each cluster included in each sentence group. The investigator may distinguish between a sentence that is frequently uttered by the degenerative brain function decline group in common and a sentence that is not uttered by the degenerative brain function decline group based on the characteristic sentence. Accordingly, the operating process of steps S410 to S460 of FIG. 6 may be said to be the explainable model that assists the prediction model according to an embodiment of the present disclosure.

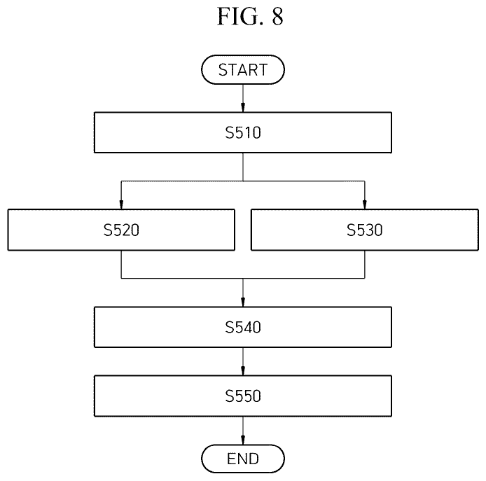

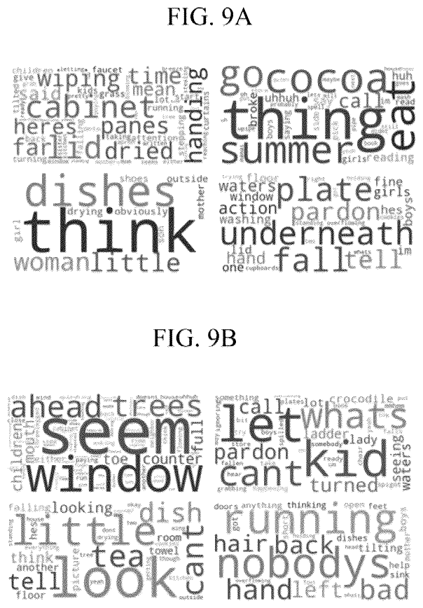

FIG. 8 is a flowchart for describing a method of displaying keywords of a normal group and a degenerative brain function decline group (hereinafter abbreviated as a “keyword display method”) through the explainable model for the prediction of a degenerative brain function decline and a cognitive impairment according to an embodiment of the present disclosure. Furthermore, FIGS. 9A and 9B are diagrams illustrating examples of keywords of a normal group and a degenerative brain function decline group, which are derived through a similarity-based comparison and a dissimilarity-based comparison.

The embodiment of FIG. 6 relates to a method of executing the explainable model, which is focused on a sentence. In contrast, the embodiment of FIG. 8 relates to a method of executing the explainable model, which is focused on a keyword. That is, the method according to the embodiment of FIG. 8 may be said to be a method of describing keywords so that a keyword that is frequently used by the degenerative brain function decline group in common and a keyword that is frequently used by the normal group can be distinguished.

The keyword display method may be performed by the prediction apparatus 100. An embodiment in which the prediction apparatus 100 performs steps included in the keyword display method, for convenience's sake, is described.

Referring to FIG. 8, the keyword display method according to an embodiment of the present disclosure includes steps S510 to S550. The keyword display method illustrated in FIG. 8 is based on an embodiment. The steps of the keyword display method according to an embodiment of the present disclosure are not limited to the embodiment illustrated in FIG. 8, and a component may be added, changed, or deleted, if necessary.

The keyword display method of FIG. 8 is executed after the prediction method of FIG. 3 or 5 is executed.

Before the keyword display method of FIG. 8 is executed, the processor 110 trains the prediction model by using the learning data (D_{img}, D_{vc}, and R_{cls} in FIG. 2) of the picture description task according to the embodiment of FIG. 3 or 5, and extracts a learning data sample that accords with an answer based on the prediction model (i.e., a mode corresponding to the best epoch) having the best prediction performance and the results of the classification of the classifier. The keyword display method of FIG. 8 is performed based on a learning data sample that accords with an answer on the basis of the prediction model having the highest prediction performance.

Step S510 is the same as step S410 in the embodiment (i.e., the representative sentence display method) of FIG. 6, and a description thereof is omitted.

Step S520 is a similarity-based comparison step. Step S530 is a dissimilarity-based comparison step.

Steps S520 and S530 include contents that are performed in steps S420 and S430 of FIG. 6. In this case, in step S520, a similarity low-rank sentence group for the representative embedding vector of each group is also derived. In step S530, a similarity high-rank sentence group for the representative embedding vector of a counterpart group is also derived. That is, in steps S520 and S530, the processor 110 derives both the similarity high-rank sentence group and the similarity low-rank sentence group for the representative embedding vectors of each group and the counterpart group. In other words, the processor 110 also extracts a set of irrelevant sentences in a similarity-based comparison and a dissimilarity-based comparison. For reference, S_{AD, ˜}, S_{HC, ˜}, [symbol] , and [symbol] that are derived in the embodiment of FIG. 6 are relevant sentence sets.

Hereinafter, a process of extracting an irrelevant sentence set in steps S520 and S530 is described.

For example, in step S520 (similarity-based comparison), the processor 110 compares the representative embedding vector of the same group and the sentence embedding vector of the same group, and may construct an irrelevant sentence set from a cosine similarity lowest rank up to a low-rank 5% sentence in ascending power.

In this specification, an irrelevant sentence set of a normal group that is derived by the similarity-based comparison is denoted as \tilde{S}_{HC,˜}. An irrelevant sentence set of a degenerative brain function decline group that is derived by the similarity-based comparison is denoted as \tilde{S}_{AD,˜}. That is, \tilde{S}_{HC,˜} is the similarity low-rank sentence group of the normal group for the representative embedding vector of the normal group. \tilde{S}_{AD,˜} is the similarity low-rank sentence group of the degenerative brain function decline group for the representative embedding vector of the degenerative brain function decline group.

Furthermore, for example, in step S530 (dissimilarity-based comparison), the processor 110 compares the representative embedding vector of a counterpart group and the sentence embedding vector of the counterpart group, and may construct an irrelevant sentence set including sentences from a cosine similarity highest rank up to a high-rank 5% in descending power.

In this specification, an irrelevant sentence set of the normal group that is derived by the dissimilarity-based comparison is denoted as [symbol] . An irrelevant sentence set of a degenerative brain function decline group that is derived by the dissimilarity-based comparison is denoted as [symbol] . That is, [symbol] is the similarity high-rank sentence group of the normal group for the representative embedding vector of the degenerative brain function decline group. [symbol] the similarity high-rank sentence group of the degenerative brain function decline group for the representative embedding vector of the normal group.

Sentence groups derived in steps S520 and S530 are arranged in Table 1. In Table 1, relevant sentence sets are S_{AD, ˜}, S_{HC,˜}, [symbol] , and [symbol] , and irrelevant sentence sets are \tilde{S}_{HC,˜}, \tilde{S}_{AD,˜}, [symbol] , and [symbol] .

TABLE 1

| | Comparison target group | | | |
|---|---|---|---|---|
| | | | Degenerative brain | |
| | Normal group | | function decline group | |
| | Similarity | Similarity | Similarity | Similarity |
| Representative | high-rank | low-rank | high-rank | low-rank |
| embedding | sentence | sentence | sentence | sentence |
| vector | group | group | group | group |
| Representative | S_{HC, ~} | \tilde{S}_{HC, ~} | [symbol] | [symbol] |
| embedding | | | | |
| vector of | | | | |
| normal group | | | | |
| Representative | [symbol] | [symbol] | S_{AD, ~} | \tilde{S}_{AD, ~} |
| embedding | | | | |
| vector of | | | | |
| degenerative | | | | |
| brain function | | | | |
| decline group | | | | |

When each sentence group is derived through the similarity-based comparison S520 and the dissimilarity-based comparison S530, step S540 is performed.

Step S540 is a step of extracting words that appear exclusively to each other in each sentence group.

The processor 110 may extract words that appear exclusively to each other between the sentence groups of Table 1, which are derived in steps S520 and S530, may generate a word cloud image based on frequency of the extracted words, and may then display the generated word cloud image through the output interface device 160. The word extracted by the prediction apparatus 100 may be displayed in the form of a table or a graph.

For example, in step S540, examples of the sentence groups, that is, exclusive comparison targets, and keywords derived based on the results of the comparison may be indicated like Table 2.

TABLE 2

| | Word | | |
|---|---|---|---|
| | extraction | Comparison | |
| | target group | target group | Examples of keywords |
| (1) | S_{HC, ~} | S_{AD, ~} | Top left in FIG. 9A |
| (2) | S_{AD, ~} | S_{HC, ~} | Top right in FIG. 9A |
| (3) | [symbol] | [symbol] | Top left in FIG. 9B |
| (4) | [symbol] | [symbol] | Top right in FIG. 9B |
| (5) | \tilde{S}_{HC, ~} | \tilde{S}_{AD, ~} | Bottom left in FIG. 9A |
| (6) | \tilde{S}_{AD, ~} | \tilde{S}_{HC, ~} | Bottom right in FIG. 9A |
| (7) | [symbol] | [symbol] | Bottom left in FIG. 9B |
| (8) | [symbol] | [symbol] | Bottom right in FIG. 9B |

(1) and (2) in Table 2 are cases in which words that appear exclusively to each other between S_{AD,˜} and S_{HC,˜} are extracted. The top left in FIG. 9A illustrates words that exclusively appear between S_{AD,˜} and S_{HC,˜} and that belong to the group S_{HC,˜}. The top right in FIG. 9A illustrates words that exclusively appear between S_{AD,˜} and S_{HC,˜} and that belong to the group S_{AD,˜}.

(3) and (4) in Table 2 are cases in which words that appear exclusively to each other between [symbol] and [symbol] are extracted. The top left in FIG. 9B illustrates words that exclusively appear between [symbol] and [symbol] and that belong to the group [symbol] . The top right in FIG. 9B illustrates words that exclusively appear between [symbol] and [symbol] and that belong to the group [symbol] .

(5) and (6) in Table 2 are cases in which words that appear exclusively to each other between \tilde{S}_{AD,˜} and \tilde{S}_{HC,˜} are extracted. The bottom left in FIG. 9A illustrates words that exclusively appear between \tilde{S}_{AD,˜} and \tilde{S}_{HC,˜} and that belong to the group \tilde{S}_{HC,˜}. The bottom right in FIG. 9A illustrates words that exclusively appear between \tilde{S}_{AD,˜} and \tilde{S}_{HC,˜} and that belong to the group \tilde{S}_{AD,˜}.

(7) and (8) in Table 2 are cases in which words that appear exclusively to each other between [symbol] , and [symbol] are extracted. The bottom left in FIG. 9B illustrates words that exclusively appear between [symbol] and [symbol] and that belong to the group [symbol] . The bottom right in FIG. 9B illustrates words that exclusively appear between [symbol] and [symbol] and that belong to the group [symbol] .

The processor 110 may extract words that exclusively appears in a word extraction target group by comparing a word in the word extraction target group of Table 2 and a word in a comparison target group, may construct a word cloud by using the extracted words, and may then display the word cloud through the output interface device 160 as illustrated in FIGS. 9A and 9B(S550).

The top left in each of FIGS. 9A and 9B illustrates keywords (relevant) of the normal group HC, which help in distinguishing the normal group from the degenerative brain function decline group. The top right in each of FIGS. 9A and 9B illustrates keywords (relevant) of the degenerative brain function decline group AD, which help in distinguishing the degenerative brain function decline group from the normal group.

Furthermore, the bottom left in each of FIGS. 9A and 9B illustrates keywords (irrelevant) of the normal group HC, which do not help in distinguishing between the normal group HC and the degenerative brain function decline group AD. The bottom right in each of FIGS. 9A and 9B illustrates keywords (irrelevant) of the degenerative brain function decline group AD, which do not help in distinguishing between the degenerative brain function decline group AD and the normal group HC.

That is, the embodiment of FIG. 8 proposes a method of describing a difference between the representative keywords of the normal group and the degenerative brain function decline group through the explainable model for the prediction of a degenerative brain function decline and a cognitive impairment, which is constructed through the training of the GNN.

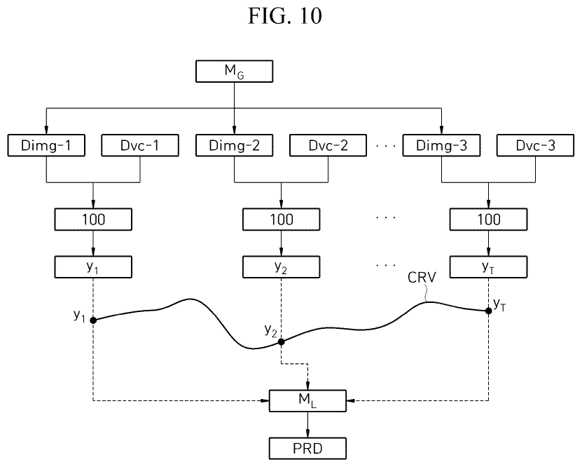

FIG. 10 is a diagram for describing a longitudinal analysis method for the early prediction of a degenerative brain function decline and a cognitive impairment.

For longitudinal analysis, one subject needs to perform a picture description task several times at regular time intervals (e.g., several months or several years). If tests are performed by using the same picture when the picture description task is performed several times, independence between experiments cannot be guaranteed. Accordingly, it is necessary to present various pictures. That is, if a subject repeatedly performs a description of the same picture, the subject may gradually describe a picture in detail and clearly. In order to remove such a possibility, it is necessary to generate various target images D_{img}-1, D_{img}-2, D_{img}-3, . . . through a generation model (generative model) M_{G} and to present the various target images to a subject.

The prediction apparatus 100 may use various models as the generation model M_{G}. For example, the processor 110 may generate a target image by using DALLE3 of ChatGPT. Specifically, the processor 110 inputs a text prompt in which sentences including various elements are listed to the generation model M_{G} so that the generation model M_{G} can generate a target image including several situations. For example, the text prompt that is input to the generation model M_{G} by the processor 110 may be constructed as follows.

[Example of Text Prompt that is Input to Generation Model]

Please consider the following description and draw a picture; The view of a kitchen can be seen. There are a mother, a son, and a daughter. The mother is wiping the water from a dish with a dishcloth in front of a sink. The son goes up on a stool, opens the lid of a cookie bowl in the top cabinet of the kitchen, and holds a cookie in his hand. The stool is about to fall. The mother is not aware of the situation. The daughter is reaching for cookies next to her brother. A sink is not turned off, so the water is overflowing the sink because the sink is full. The overflown water is falling toward the floor. There is a kitchen window over the sink, and curtains are fluttering in the wind. Outside the kitchen window, you can see a neighbor's house, a lawn is spread out in front of the neighbor's house, and you can see trees in the distance.

When the processor 110 inputs the text prompt to the generation model M_{G}, the generation model M_{G} may generate target images illustrated in FIG. 4A.

The generated target images are presented to a subject. The subject is required to describe situations within the target images. Different target images generated as various prompts are used every experiment. The subject describes each of the target images D_{img}-1, D_{img}-2, and D_{img}-3 at time intervals. In this process, utterance speech data D_{vc}-1, D_{vc}-2, and D_{vc}-3 of the subject are collected.

The prediction apparatus 100 receives the target images and the utterance speech data of the subject, and calculates the probability (y) that the subject will belong to the normal group based on a vision language model and a graph neural network (refer to the prediction methods in FIGS. 3 and 5).

The prediction apparatus 100 may obtain the normal probability (y) of the subject through the prediction method based on a graph neural network (refer to Equation 3). If such a process is performed several times at time intervals, time-series data CRV having a normal probability (y), such as y₁, y₂, . . . , y_{T}, are obtained. This is the results of the execution of a total of T picture description tasks. The prediction apparatus 100 may generate a model M_{L} (hereinafter referred to as a “longitudinal analysis model”) that determines whether a subject becomes close to a degenerative brain function decline group or continues to maintain a healthy cognition ability (i.e., a normal group) by training a machine learning model or a transformer model based on learning data (i.e., a target image, utterance speech data, and time-series data of the normal probability) that are generated through such a process. Models which may be used as the longitudinal analysis model are various. A gradient boosting decision tree (GBDT) method, such as LightGBM or XGBoost, or a random forest model may be used as the machine learning model. A transformer or an LSTM may be used as a deep learning model. The longitudinal analysis model receives the target image D_{img}, the utterance speech data D_{vc} of a subject, and time-series data having the normal probability (y) calculated by the prediction model. The subject predicts a probability and/or a period (PRD) in which the subject will reach a degenerative brain function decline and a degenerative brain function decline (the results of early prediction).

The prediction methods of FIGS. 3 and 5, the representative sentence display method of FIG. 6, and the keyword display method of FIG. 8 have been described with reference to the flowcharts. For a simple description, the methods each have been illustrated and described as a series of blocks, but the present disclosure is not limited to the sequence of the blocks, and some blocks may be performed in a sequence different from or simultaneously with that of other blocks, which has been illustrated and described in this specification. Various other branches, flow paths, and sequences of blocks which achieve the same or similar results may be implemented. Furthermore, all the blocks illustrated in order to implement the method described in this specification may not be required.

In the descriptions given with reference to FIGS. 3, 5, 6, and 8, each of the steps may be further divided into additional steps or the steps may be combined into smaller steps depending on an implementation example of the present disclosure. Furthermore, some of the steps may be omitted, if necessary, and the sequence of the steps may be changed. For example, the contents of FIG. 3 may be applied to the contents of FIG. 5. The contents of FIGS. 3 and 5 may be applied to the contents of FIGS. 6, 8, and 10. Furthermore, the contents of FIG. 6 may be applied to the contents of FIG. 8.

Although the present disclosure has been described with reference to the preferred embodiments, those skilled in the art may understand that the present disclosure may be modified and changed in various ways without departing from the spirit and scope of the present disclosure written in the claims.

### DESCRIPTION OF REFERENCE NUMERALS

100: apparatus for predicting degenerative brain function decline and cognitive impairment
110: processor 120: communication device
130: memory 140: storage device
150: input interface device
160: output interface device 170: bus
   S210: Receive target image of picture description task
   S215: Color image?
   S220: Coloring
   S225: Generate sub-image through image cropping
   S230: Generate sub-image embedding vector by using vision language model (VLM)
   S240: Receive utterance speech data of subject
   S245: Generate utterance text through automatic transcription
   S250: Segment utterance text in sentence unit
   S255: Generate sentence embedding vector by using VLM
   S260: Calculate similarity between sub-image and sentence by using VLM
   S265: Generate bipartite graph including sub-image node and sentence node
   S270: Generate graph-level embedding vector by using graph convolution neural network (GCN)
   S275: Classify subject as degenerative brain function decline group or normal group
   S310: Receive target image of picture description task
   S315: Color image?
   S320: Coloring
   S325: Generate sub-image through image cropping
   S330: Generate sub-image embedding vector by using vision language model (VLM)
   S340: Receive utterance speech data of subject
   S345: Generate utterance text through automatic transcription
   S350: Segment utterance text in sentence unit
   S355: Generate sentence embedding vector by using VLM
   S360: Calculate similarity between sub-image and sentence by using VLM
   S365: Generate bipartite graph including sub-image node and sentence node
   S370: Generate graph-level embedding vector by using graph convolution neural network (GCN)
   S375: Extract speech feature from utterance speech data
   S380: Generate speech embedding vector by using learning model
   S385: Extract text feature by using language model
   S390: Generate text embedding vector by using learning model
   S395: Classify subject as degenerative brain function decline group or normal group
   S410: Generate representative embedding vectors of degenerative brain function decline group and normal group
   S420: [similarity-based comparison] Extract similarity high-rank sentence for representative embedding vector of each group
   S430: [dissimilarity-based comparison] Extract similarity low-rank sentence for representative embedding vector of counterpart group
   S440: Obtain text embedding vector of extracted sentence
   S450: Group extracted sentence through clustering
   S460: Display representative sentence of each of clusters of degenerative brain function decline group and normal group
   S510: Generate representative embedding vectors of degenerative brain function decline group and normal group
   S520: [similarity-based comparison] Derive similarity high-rank sentence group and similarity low-rank sentence group for representative embedding vector of each group
   S530: [dissimilarity-based comparison] Derive similarity high-rank sentence group and similarity low-rank sentence group for representative embedding vector of counterpart group
   S540: Extract words that appear exclusively to each other in each group
   S550: Display extracted words so that keywords between degenerative brain function decline group and normal group are different

What is claimed is:

1. A method performed by an apparatus for predicting a degenerative brain function decline and a cognitive impairment, the method comprising:
receiving a target image of a picture description task and utterance speech data of a subject for the target image;
generating a sub-image embedding vector that is an embedding vector of a sub-image of the target image;
extracting one or more sentences by segmenting utterance text that is generated by transcribing the utterance speech data in a sentence unit and generating a sentence embedding vector that is an embedding vector of the sentence; and
calculating similarity between the sub-image and the sentence by using a vision language model and determining that the subject corresponds to any one of a degenerative brain function decline and cognitive impairment group and a normal group based on the sub-image embedding vector, the sentence embedding vector, and the similarity.

2. The method of claim 1, wherein the generating of the sub-image embedding vector comprises generating the sub-image embedding vector by using the vision language model.

3. The method of claim 1, wherein the generating of the sentence embedding vector comprises generating the sentence embedding vector by using the vision language model.

4. The method of claim 1, wherein the determining of that the subject corresponds to any one of the degenerative brain function decline and cognitive impairment group and the normal group comprises:
calculating the similarity between the sub-image and the sentence by using the vision language model;
generating a bipartite graph comprising a sub-image node corresponding to the sub-image embedding vector and a sentence node corresponding to the sentence embedding vector, wherein a weight of an edge that connects the sub-image node and the sentence node is set in the bipartite graph based on the similarity;
inputting the bipartite graph to a graph neural network and generating a graph-level embedding vector through information propagation; and
calculating a probability that the subject is to belong to the normal group by inputting the graph-level embedding vector to a classifier based on a pre-trained artificial neural network and classifying the subject as any one group of the degenerative brain function decline and cognitive impairment group and the normal group based on the probability.

5. The method of claim 4, wherein the graph neural network is a graph convolution neural network.

6. The method of claim 4, further comprising:
extracting a speech feature of the subject from the utterance speech data; and
generating a speech embedding vector based on the speech feature,
wherein the classifying of the subject comprises calculating the probability by inputting the graph-level embedding vector and the speech embedding vector to the classifier.

7. The method of claim 4, further comprising:
extracting a text feature by inputting the utterance text to a language model; and
generating the text embedding vector based on the text feature,
wherein the classifying of the subject comprises calculating the probability by inputting the graph-level embedding vector and the text embedding vector to the classifier.

8. The method of claim 4, further comprising:
generating a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed;
calculating first similarity between the first representative embedding vector and the sentence embedding vector of the degenerative brain function decline and cognitive impairment group and generating a similarity high-rank sentence group by grouping sentence embedding vectors corresponding to a predetermined high-rank percentage, among all of the sentence embedding vectors of the degenerative brain function decline and cognitive impairment group, based on the first similarity;
calculating second similarity between the second representative embedding vector and the sentence embedding vector of the degenerative brain function decline and cognitive impairment group and generating a similarity low-rank sentence group by grouping sentence embedding vectors corresponding to a predetermined low-rank percentage, among all of the sentence embedding vectors of the degenerative brain function decline and cognitive impairment group, based on the second similarity; and
selecting representative sentences corresponding to the similarity high-rank sentence group and the similarity low-rank sentence group, respectively, based on a predetermined reference and displaying the representative sentences through an output interface device.

9. The method of claim 4, further comprising:
generating a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed;
calculating first similarity between the first representative embedding vector and the sentence embedding vector of the degenerative brain function decline and cognitive impairment group, generating a first relevant sentence set by grouping sentence embedding vectors corresponding to a predetermined high-rank percentage, among all of the sentence embedding vectors of the degenerative brain function decline and cognitive impairment group, based on the first similarity, and generating a first irrelevant sentence set by grouping sentence embedding vectors corresponding to a predetermined low-rank percentage, among all of the sentence embedding vectors of the degenerative brain function decline and cognitive impairment group, based on the first similarity;
calculating second similarity between the second representative embedding vector and the sentence embedding vector of the degenerative brain function decline and cognitive impairment group, generating a second relevant sentence set by grouping sentence embedding vectors corresponding to a predetermined low-rank percentage, among all of the sentence embedding vectors of the degenerative brain function decline and cognitive impairment group, based on the second similarity, and generating a second irrelevant sentence set by grouping sentence embedding vectors corresponding to a predetermined high-rank percentage, among all of the sentence embedding vectors of the degenerative brain function decline and cognitive impairment group, based on the second similarity;
calculating third similarity between the second representative embedding vector and the sentence embedding vector of the normal group, generating a third relevant sentence set by grouping sentence embedding vectors corresponding to a predetermined high-rank percentage, among all of the sentence embedding vectors of the normal group, based on the third similarity, and generating a third irrelevant sentence set by grouping sentence embedding vectors corresponding to a predetermined low-rank percentage, among all of the sentence embedding vectors of the normal group, based on the third similarity;
calculating fourth similarity between the first representative embedding vector and the sentence embedding vector of the normal group, generating a fourth relevant sentence set by grouping sentence embedding vectors corresponding to a predetermined low-rank percentage, among all of the sentence embedding vectors of the normal group, based on the fourth similarity, and generating a fourth irrelevant sentence set by grouping sentence embedding vectors corresponding to the predetermined high-rank percentage, among all of the sentence embedding vectors of the normal group, based on the fourth similarity;
selecting a word not included in the third relevant sentence set, among words included in the first relevant sentence set, and setting the selected word as a first keyword, selecting a word not included in the fourth relevant sentence set, among words included in the second relevant sentence set, and setting the selected word as a second keyword, selecting a word not included in the third irrelevant sentence set, among words included in the first irrelevant sentence set, and setting the selected word as a third keyword, selecting a word not included in the fourth irrelevant sentence set, among words included in the second irrelevant sentence set, and setting the selected word as a fourth keyword; and
outputting the first keyword and the second keyword as keyword that help in determining the degenerative brain function decline and cognitive impairment group and outputting the third keyword and the fourth keyword as keywords that do not help in determining the degenerative brain function decline and cognitive impairment group.

10. The method of claim 4, further comprising:
calculating the probability for an identical subject with respect to different target images at regular time intervals during a predetermined period by a predetermined number of times; and
inputting the probability calculated during the predetermined period to a pre-trained longitudinal analysis model and determining that the subject is to belong to the degenerative brain function decline and cognitive impairment group after a predetermined period based on an output of the longitudinal analysis model.

11. An apparatus for predicting a degenerative brain function decline and a cognitive impairment, the apparatus comprising:
a processor; and
memory in which one or more instructions executed by the processor are stored,
wherein the one or more instructions comprise:
an instruction to receive a target image of a picture description task and utterance speech data of a subject for the target image;
an instruction to generate a sub-image embedding vector that is an embedding vector of a sub-image of the target image;
an instruction to extract one or more sentences by segmenting utterance text that is generated by transcribing the utterance speech data in a sentence unit and to generate a sentence embedding vector that is an embedding vector of the sentence; and
an instruction to calculate similarity between the sub-image and the sentence by using a vision language model and to determine that the subject corresponds to any one of a degenerative brain function decline and cognitive impairment group and a normal group based on the sub-image embedding vector, the sentence embedding vector, and the similarity.

12. The apparatus of claim 11, wherein the instruction to generate the sub-image embedding vector comprises an instruction to generate the sub-image embedding vector by using the vision language model.

13. The apparatus of claim 11, wherein the instruction to generate the sentence embedding vector comprises an instruction to generate the sentence embedding vector by using the vision language model.

14. The apparatus of claim 11, wherein the instruction to determine that the subject corresponds to any one of the degenerative brain function decline and cognitive impairment group and the normal group comprises:
an instruction to calculate the similarity between the sub-image and the sentence by using the vision language model;
an instruction to generate a bipartite graph comprising a sub-image node corresponding to the sub-image embedding vector and a sentence node corresponding to the sentence embedding vector, wherein a weight of an edge that connects the sub-image node and the sentence node is set in the bipartite graph based on the similarity;
an instruction to input the bipartite graph to a graph neural network and to generate a graph-level embedding vector through information propagation; and
an instruction to calculate a probability that the subject is to belong to the normal group by inputting the graph-level embedding vector to a classifier based on a pre-trained artificial neural network and to classify the subject as any one group of the degenerative brain function decline and cognitive impairment group and the normal group based on the probability.

15. The apparatus of claim 14, wherein the graph neural network is a graph convolution neural network.

16. The apparatus of claim 14, wherein the one or more instructions further comprise:
an instruction to extract a speech feature of the subject from the utterance speech data; and
an instruction to generate a speech embedding vector based on the speech feature,
wherein the instruction to classify the subject comprises an instruction to calculate the probability by inputting the graph-level embedding vector and the speech embedding vector to the classifier.

17. The apparatus of claim 14, wherein the one or more instructions further comprise:
an instruction to extract a text feature by inputting the utterance text to a language model; and
an instruction to generate the text embedding vector based on the text feature,
wherein the instruction to classify the subject comprises an instruction to calculate the probability by inputting the graph-level embedding vector and the text embedding vector to the classifier.

18. The apparatus of claim 14, wherein the one or more instructions further comprise:
an instruction to generate a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed;
an instruction to calculate first similarity between the first representative embedding vector and the sentence embedding vector of the degenerative brain function decline and cognitive impairment group and to generate a similarity high-rank sentence group by grouping sentence embedding vectors corresponding to a predetermined high-rank percentage, among all of the sentence embedding vectors of the degenerative brain function decline and cognitive impairment group, based on the first similarity;
an instruction to calculate second similarity between the second representative embedding vector and the sentence embedding vector of the degenerative brain function decline and cognitive impairment group and generating a similarity low-rank sentence group by grouping sentence embedding vectors corresponding to a predetermined low-rank percentage, among all of the sentence embedding vectors of the degenerative brain function decline and cognitive impairment group, based on the second similarity; and
an instruction to select representative sentences corresponding to the similarity high-rank sentence group and the similarity low-rank sentence group, respectively, based on a predetermined reference and displaying the representative sentences through an output interface device.

19. The apparatus of claim 14, wherein the one or more instructions further comprise:
an instruction to generate a first representative embedding vector that is a representative embedding vector of the degenerative brain function decline and cognitive impairment group and a second representative embedding vector that is a representative embedding vector of the normal group based on sentence embedding vectors of the degenerative brain function decline and cognitive impairment group and the normal group on which information propagation has been completed;
an instruction to calculate first similarity between the first representative embedding vector and the sentence embedding vector of the degenerative brain function decline and cognitive impairment group, to generate a first relevant sentence set by grouping sentence embedding vectors corresponding to a predetermined high-rank percentage, among all of the sentence embedding vectors of the degenerative brain function decline and cognitive impairment group, based on the first similarity, and to generate a first irrelevant sentence set by grouping sentence embedding vectors corresponding to a predetermined low-rank percentage, among all of the sentence embedding vectors of the degenerative brain function decline and cognitive impairment group, based on the first similarity;
an instruction to calculate second similarity between the second representative embedding vector and the sentence embedding vector of the degenerative brain function decline and cognitive impairment group, to generate a second relevant sentence set by grouping sentence embedding vectors corresponding to a predetermined low-rank percentage, among all of the sentence embedding vectors of the degenerative brain function decline and cognitive impairment group, based on the second similarity, and to generate a second irrelevant sentence set by grouping sentence embedding vectors corresponding to a predetermined high-rank percentage, among all of the sentence embedding vectors of the degenerative brain function decline and cognitive impairment group, based on the second similarity;
an instruction to calculate third similarity between the second representative embedding vector and the sentence embedding vector of the normal group, to generate a third relevant sentence set by grouping sentence embedding vectors corresponding to a predetermined high-rank percentage, among all of the sentence embedding vectors of the normal group, based on the third similarity, and to generate a third irrelevant sentence set by grouping sentence embedding vectors corresponding to a predetermined low-rank percentage, among all of the sentence embedding vectors of the normal group, based on the third similarity;
an instruction to calculate fourth similarity between the first representative embedding vector and the sentence embedding vector of the normal group, to generate a fourth relevant sentence set by grouping sentence embedding vectors corresponding to a predetermined low-rank percentage, among all of the sentence embedding vectors of the normal group, based on the fourth similarity, and to generate a fourth irrelevant sentence set by grouping sentence embedding vectors corresponding to the predetermined high-rank percentage, among all of the sentence embedding vectors of the normal group, based on the fourth similarity;
an instruction to select a word not included in the third relevant sentence set, among words included in the first relevant sentence set, and to set the selected word as a first keyword, to select a word not included in the fourth relevant sentence set, among words included in the second relevant sentence set, and to set the selected word as a second keyword, to select a word not included in the third irrelevant sentence set, among words included in the first irrelevant sentence set, and to set the selected word as a third keyword, to select a word not included in the fourth irrelevant sentence set, among words included in the second irrelevant sentence set, and to set the selected word as a fourth keyword; and
an instruction to output the first keyword and the second keyword as keyword that help in determining the degenerative brain function decline and cognitive impairment group and to output the third keyword and the fourth keyword as keywords that do not help in determining the degenerative brain function decline and cognitive impairment group.

20. The apparatus of claim 14, wherein the one or more instructions further comprise:
an instruction to calculate the probability for an identical subject with respect to different target images at regular time intervals during a predetermined period by a predetermined number of times; and
an instruction to input the probability calculated during the predetermined period to a pre-trained longitudinal analysis model and to determine that the subject is to belong to the degenerative brain function decline and cognitive impairment group after a predetermined period based on an output of the longitudinal analysis model.
